(19) United States
(12) Patent Application Publication
(10) Pub. No.: US 2026/0166116 A1
(43) Pub. Date: Jun. 18, 2026

(54) CANCER IMMUNOTHERAPY METHODS

(71) Applicant: Ohio State Innovation Foundation, Columbus, OH (US)

(72) Inventors: Zihai LI, Columbus, OH (US); No Joon SONG, Columbus, OH (US)

(21) Appl. No.: 19/128,717

(22) PCT Filed: Nov. 9, 2023
(86) PCT No.: PCT/US2023/079247

§ 371 Date: May 9, 2025

Related U.S. Application Data
(60) Provisional application No. 63/382,936, filed on Nov. 9, 2022.

(51) Int. Cl. A61K 38/00; A61K 9/00; A61K 40/11

(52) U.S. Cl. CPC A61K 38/00; A61K 9/0019; A61K 40/11

(57) ABSTRACT

The present disclosure relates compositions and methods for treating, preventing, reducing, and/or ameliorating a cancer.

### RELATED APPLICATION

This PCT application claims priority to, and the benefit of, U.S. Provisional Patent Application No. 63/382,936, filed Nov. 9, 2022, entitled “EXERCISE-INDUCED HORMONE IRISIN POTENTIATES ANTI-TUMOR IMMUNITY VIA HINDERING INTEGRIN ALPHA V/TGF-BETA AXIS IN TUMOR MICROENVIRONMENT,” which is incorporated by reference herein in its entirety.

### FIELD

The present disclosure relates compositions and methods for treating, preventing, reducing, and/or ameliorating a cancer.

### BACKGROUND

Growing evidence shows that exercise prevents cancer development, enhances efficacy of anti-tumor therapies, and prevents recurrence after treatment. Exercise-induced hormones including insulin, cortisol, human growth hormone, testosterone, and epinephrine have been studied in this context, especially relating to CD8⁺ T cell function. Exercise also induces secretion of irisin from muscle cells, but the role of irisin in tumor immunology has not been previously reported. Irisin suppresses tumor growth in multiple pre-clinical models through T cell-dependent mechanisms. Irisin treatment reduced accumulation of regulatory T cells (Tregs), leading to less dysfunction in the CD8⁺ T cell population in the tumor microenvironment (TME).

Given the limitations of current immunotherapies against cancer, there is a need to improve treatment methods to treat cancer and enhance the efficacy of anti-tumor therapies.

### SUMMARY

The present disclosure provides methods of using irisin compositions to treat, prevent, decrease, reduce, and/or ameliorate cancer.

In one aspect, disclosed herein are methods of treating, reducing, decreasing, inhibiting, ameliorating, and/or preventing a cancer and/or metastasis (including, but not limited to colon carcinoma, melanoma, urothelial carcinoma, and lung cancer) in a subject, the method comprising administering a pharmaceutically effective amount of a composition comprising irisin and a pharmaceutically acceptable carrier (including, but not limited to an excipient, a diluent, a salt, a buffer, a stabilizer, a lipid, an emulsion, and a nanoparticle) wherein the composition prevents TGF-β activation in the subject relative to an untreated control.

In some aspects, disclosed herein are methods of treating, reducing, decreasing, inhibiting, ameliorating, and/or preventing a cancer and/or metastasis of any preceding aspect wherein irisin targets an integrin αvβ5 complex. In some aspects, the methods of treating, reducing, decreasing, inhibiting, ameliorating, and/or preventing a cancer and/or metastasis comprises a composition that prevents release and activation of TGF-β and/or reduces tumor growth in the subject in the subject relative to an untreated control. Also disclosed herein are methods of treating, reducing, decreasing, inhibiting, ameliorating, and/or preventing a cancer and/or metastasis of any preceding aspect wherein the composition causes accumulation of functional T cells including, but not limited to a functional CD4⁺ T cells or a functional CD8⁺ T cells (such as, for example, effector CD8 T cells, central memory CD8 T cells, and peripheral memory CD8 T cells) in the subject relative to an untreated control.

In some aspects, disclosed herein are methods of treating and/or preventing a cancer of any preceding aspect further comprising administering an immune checkpoint inhibitor including, but not limited to antibodies that block PD-1 (such as, for example, Nivolumab (BMS-936558 or MDX1106), pembrolizumab, cemiplimab, CT-011, MK-3475), PD-L1 (such as, for example, atezolizumab, avelumab, durvalumab, MDX-1105 (BMS-936559), MPDL3280A, or MSB0010718C), PD-L2 (such as, for example, rHIgM12B7), CTLA-4 (such as, for example, Ipilimumab (MDX-010), Tremelimumab (CP-675,206)), IDO, B7-H3 (such as, for example, MGA271, MGD009, omburtamab), B7-H4, B7-H3, T cell immunoreceptor with Ig and ITIM domains (TIGIT) (such as, for example BMS-986207, OMP-313M32, MK-7684, AB-154, ASP-8374, MTIG7192A, or PVSRIPO), CD96, B- and T-lymphocyte attenuator (BTLA), V-domain Ig suppressor of T cell activation (VISTA) (such as, for example, JNJ-61610588, CA-170), TIM3 (such as, for example, TSR-022, MBG453, Sym023, INCAGN2390, LY3321367, BMS-986258, SHR-1702, RO7121661), LAG-3 (such as, for example, BMS-986016, LAG525, MK-4280, REGN3767, TSR-033, BI754111, Sym022, FS118, MGD013, and Immutep) or a combination thereof.

In one aspect, disclosed herein are methods of slowing, inhibiting, reducing, decreasing, and/or preventing tumor growth factor β (TGF-β) activation in a tumor (including, but not limited to tumors derived from colon carcinoma, melanoma, urothelial carcinoma, and lung cancer), the method comprising administering a composition comprising irisin and a pharmaceutically acceptable carrier, including, but not limited to an excipient, a diluent, a salt, a buffer, a stabilizer, a lipid, an emulsion, and a nanoparticle, to a subject with said tumor, and detecting a decreased amount of an activated TGF-β in the tumor relative to an amount of the activated TGF-β in a control tumor. In some embodiments, the method of slowing, inhibiting, reducing, decreasing, and/or preventing tumor growth factor β (TGF-β) activation treats and/or prevents the cancer.

Also disclosed herein are methods of slowing, inhibiting, reducing, decreasing, and/or preventing tumor growth factor β (TGF-β) activation of any preceding aspect, wherein irisin targets an integrin αvβ5 complex. In some aspects, the composition prevents, reduces, and/or inhibits release and/or activation of TGF-β. Also disclosed herein are methods of slowing, inhibiting, reducing, decreasing, and/or preventing tumor growth factor β (TGF-β) activation of any preceding aspect, wherein the composition reduces tumor growth and/or causes accumulation of functional T cells (such as, for example, CD4⁺ T cells or a functional CD8⁺ T cells (such as, for example, effector CD8 T cells, central memory CD8 T cells, and peripheral memory CD8 T cells) in the subject relative to an untreated control.

In one aspect, disclosed herein are methods of slowing, inhibiting, reducing, decreasing, and/or preventing tumor growth factor β (TGF-β) activation of any preceding aspect further comprising administering an immune checkpoint inhibitor including, but not limited to antibodies that block PD-1 (such as, for example, Nivolumab (BMS-936558 or MDX1106), pembrolizumab, cemiplimab, CT-011, MK-3475), PD-L1 (such as, for example, atezolizumab, avelumab, durvalumab, MDX-1105 (BMS-936559), MPDL3280A, or MSB0010718C), PD-L2 (such as, for example, rHIgM12B7), CTLA-4 (such as, for example, Ipilimumab (MDX-010), Tremelimumab (CP-675,206)), IDO, B7-H3 (such as, for example, MGA271, MGD009, omburtamab), B7-H4, B7-H3, T cell immunoreceptor with Ig and ITIM domains (TIGIT) (such as, for example BMS-986207, OMP-313M32, MK-7684, AB-154, ASP-8374, MTIG7192A, or PVSRIPO), CD96, B- and T-lymphocyte attenuator (BTLA), V-domain Ig suppressor of T cell activation (VISTA) (such as, for example, JNJ-61610588, CA-170), TIM3 (such as, for example, TSR-022, MBG453, Sym023, INCAGN2390, LY3321367, BMS-986258, SHR-1702, RO7121661), LAG-3 (such as, for example, BMS-986016, LAG525, MK-4280, REGN3767, TSR-033, BI754111, Sym022, FS118, MGD013, and Immutep) or a combination thereof.

In one aspect, disclosed herein are methods of reducing, inhibiting, decreasing, and/or preventing tumor growth (including, but not limited to tumors derived from colon carcinoma, melanoma, urothelial carcinoma, and lung cancer) in a subject, the method comprising administering to a subject a pharmaceutically effective amount of a composition comprising irisin and a pharmaceutically acceptable carrier including, but not limited an excipient, a diluent, a salt, a buffer, a stabilizer, a lipid, an emulsion, and a nanoparticle, wherein the composition reduces tumor growth in the subject relative to an untreated control.

Also disclosed herein are methods of reducing, inhibiting, decreasing, and/or preventing tumor growth of any preceding aspect, wherein irisin targets an integrin αvβ5 complex. In some aspects, the composition prevents, reduces, and/or inhibits release and/or activation of TGF-β. Also disclosed herein are methods of slowing, inhibiting, reducing, decreasing, and/or preventing tumor growth factor β (TGF-β) activation of any preceding aspect, wherein the composition causes accumulation of functional T cells (such as, for example, CD4⁺ T cells or a functional CD8⁺ T cells (such as, for example, effector CD8 T cells, central memory CD8 T cells, and peripheral memory CD8 T cells) in the subject relative to an untreated control.

In one aspect, disclosed herein are methods of reducing, inhibiting, decreasing, and/or preventing tumor growth of any preceding aspect further comprising administering an immune checkpoint inhibitor including, but not limited to antibodies that block PD-1 (such as, for example, Nivolumab (BMS-936558 or MDX1106), pembrolizumab, cemiplimab, CT-011, MK-3475), PD-L1 (such as, for example, atezolizumab, avelumab, durvalumab, MDX-1105 (BMS-936559), MPDL3280A, or MSB0010718C), PD-L2 (such as, for example, rHIgM12B7), CTLA-4 (such as, for example, Ipilimumab (MDX-010), Tremelimumab (CP-675,206)), IDO, B7-H3 (such as, for example, MGA271, MGD009, omburtamab), B7-H4, B7-H3, T cell immunoreceptor with Ig and ITIM domains (TIGIT) (such as, for example BMS-986207, OMP-313M32, MK-7684, AB-154, ASP-8374, MTIG7192A, or PVSRIPO), CD96, B- and T-lymphocyte attenuator (BTLA), V-domain Ig suppressor of T cell activation (VISTA) (such as, for example, JNJ-61610588, CA-170), TIM3 (such as, for example, TSR-022, MBG453, Sym023, INCAGN2390, LY3321367, BMS-986258, SHR-1702, RO7121661), LAG-3 (such as, for example, BMS-986016, LAG525, MK-4280, REGN3767, TSR-033, BI754111, Sym022, FS118, MGD013, and Immutep) or a combination thereof.

### BRIEF DESCRIPTION OF FIGURES

The accompanying figures, which are incorporated in and constitute a part of this specification, illustrate several aspects described below.

FIGS. 1A, 1B, 1C, 1D, 1E, and 1F show that irisin reduced tumor growth in multiple pre-clinical models through a T cell-dependent mechanism. FIG. 1A shows the schematic of experimental model. FIG. 1B shows that tumor growth curve of MC38, B16F10, MB49, and LLC cells grown subcutaneously in C57BL/6J mice treated with irisin or PBS control. FIG. 1C shows the tumor growth curve of MC38 grown subcutaneously in Rag2 and TCRβδ immunocompromised mice treated with irisin or PBS control. FIG. 1D shows the MC38 tumors were collected on day 10 (after two doses of irisin or PBS) and stained with high-dimensional flow cytometry panel to assess immune cell population dynamics. Quantification of indicated cell populations within CD45⁺ immune cells. FIG. 1E shows the representative flow cytometry plots showing CD4⁺ FOXP3⁺ Treg populations from PBS-vs irisin-treated MC38 tumors. FIG. 1F shows the quantification of CD4⁺ FOXP3⁺ Tregs from day15 MC38, MB49, and LLC tumors treated with irisin or PBS. Data presented as mean±SEM. p values were calculated using two-way ANOVA in (FIG. 1B) and student t-test in (FIGS. 1D and 1F). * p<0.05; **p<0.01. 5 FIGS. 2A, 2B, 2C, 2D, 2E, 2F, and 2G show that irisin reduced suppressive phenotype in intratumoral Tregs. Tumor-infiltrating lymphocytes were collected from day 15 MC38 tumors (treated with PBS or irisin) and stained using high-dimensional flow cytometry panel to assess populational dynamics. CD4⁺ T cells were gated using web-based software OMIQ. UMAP dimension reduction and FlowSOM clustering identified 14 unique clusters with various expression patterns. 10 FIG. 2A visualized the UMAP dimension reduction and FlowSOM clustering result that was applied to CD4⁺ T cells. FIG. 2B shows the heatmap of marker expression patterns from clusters.

FIG. 2C shows the contour plots of PBS vs irisin treated CD4⁺ T cells, with arrows indicating changes to cluster frequencies. FIG. 2D shows the edgeR analysis identifying clusters with 15 significantly altered frequencies in PBS vs irisin treated tumors. FIG. 2E shows the frequencies of clusters 4 and 14 in PBS vs irisin-treated MC38 tumors. FIG. 2F shows the quantification of Treg/non-Treg CD4⁺ ratio in PBS vs irisin treated MC38 tumors. FIG. 2G shows the quantification of CTLA4⁺CD25⁺ population within CD4⁺ FOXP3⁺ Tregs in PBS vs irisin treated MC38 tumors. Data presented as mean±SEM. edgeR analysis was performed for high-dimensional flow cytometry analysis in (FIG. 2D). p-values were calculated using student t-test in (FIGS. 2E, 2F, and 2G). 20 *p<0.05; ***p<0.001.

FIGS. 3A, 3B, 3C, 3D, 3E, 3F, and 3G show that irisin treatment prevents the formation of CD8⁺ T cells with dysfunctional signatures. Tumor infiltrating lymphocytes were collected from day 15 MC38 tumors (treated with PBS or irisin) and stained using high-dimensional flow cytometry panel to assess cell population dynamics. CD8+ were gated using web-based software OMIQ. UMAP dimension reduction and FlowSOM clustering identified 16 unique clusters with various expression patterns. FIG. 3A shows the UMAP dimension reduction and FlowSOM clustering was applied to CD8⁺ T cells. FIG. 3B shows the heatmap of marker expression patterns from clusters. FIG. 3C shows the contour plots of PBS vs irisin CD8⁺ T cells, with arrows indicating changes to cluster frequencies. FIG. 3D shows the edgeR analysis identified clusters with significantly altered frequencies in PBS vs irisin treated tumors. FIG. 3E shows the expression of LAG3 and CTLA4 levels in CD8⁺ T cells from day 15 MC38 tumors. Representative flow cytometry plot and quantification shown. FIG. 3F shows the expression of TCF1 and TOX levels in CD8⁺ T cells from day 15 MC38 tumors. Representative flow cytometry plot and quantification shown. FIG. 3G shows the expression of TNF-α and IFN-γ levels in CD8⁺ T cells after ex vivo re-stimulation with PMA/Ionomycin for 4 hours. Representative flow cytometry plot and quantification shown. Data are presented as mean±SEM. edgeR analysis was performed for high-dimensional flow cytometry analysis in (FIG. 3D). p-values were calculated using student t-test in (FIGS. 3E, 3F, and 3G). **p<0.01; ***p<0.001.

FIGS. 4A, 4B, 4C, 4D, 4E, 4F, 4G, 4H, and 4I show the expression of integrin αvβ5 complex in the TME is responsible for anti-tumoral effect of irisin. FIG. 4A shows the publicly available scRNA-seq data of human lung cancer samples which was re-analyzed for cellular subsets and projected into UMAP space with cluster information. FIG. 4B shows the overlay of individual integrin expression patterns onto UMAP space from FIG. 4A. FIG. 4C shows the integrin av and β5 expression patterns (single or double positive) visualized in UMAP space. FIG. 4D shows the representative flow cytometry plot and quantification of CD11b⁺αvβ5+ cells in tumor or spleen from mice implanted with MC38 tumor. FIG. 4E shows the MC38 tumor-bearing mice treated with PE (phycoerythrin)-tagged irisin. Representative flow cytometry plot showing CD11b⁺ Irisin-PE⁺ cells in tumor. FIG. 4F shows the quantification of CD11b⁺ Irisin-PE⁺ cells in tumor and spleen. FIG. 4G shows the representative histogram and mean fluorescent intensity (MFI) quantification of integrin αvβ5 in CD11b⁺ Irisin-PE⁺ and CD11b Irisin-PE″. FIG. 4H shows the tumor growth curve of MC38 and β16F10 grown subcutaneously in WT and LysMKO mice. FIG. 4I shows the tumor growth curve of MC38 grown in WT and LysMKO treated irisin or PBS as in FIG. 1A. Data presented as mean±SEM. p-values were calculated using student t-test in (FIGS. 4D, 4F, and 4G). Two-way ANOVA was utilized for (FIGS. 4H and 4I). * p<0.05; **p<0.01; ***p<0.001.

FIGS. 5A, 5B, 5C, 5D, 5E, 5F, and 5G show that irisin treatment reduces integrin αvβ5-mediated release of active TGF-β. FIG. 5A shows the quantification of active TGF-β levels. Myeloid cells from MC38 tumors were co-incubated with L-TGF-β for 2 hours, and PBS and irisin was treated. FIG. 5B shows the quantification of active TGF-β levels utilizing myeloid cells from WT and LysMKO mice. Myeloid cells from PBS and irisin treated mouse MC38 tumors samples were co-incubated with L-TGF-β for 2 hours. FIG. 5C shows the quantification of p-Smad2/3 levels in CD8⁺ T cells from MC38 tumor samples. FIG. 5D shows the representative flow cytometry plot showing p-Smad2/3 and FOXP3 levels. FIG. 5E shows the quantification of p-Smad2/3+ cells in CD4⁺ FOXP3⁺ and CD4⁺ FOXP3⁻ populations. FIG. 5F shows the H&E staining slide and spatial data visualizing integrin av and β5 double positive (DP) and non-double positive (Non-DP) area. FIG. 5G shows the quantification of TGF-β pathway activity score in DP and Non-DP area. Data are presented as mean±SEM. p-values calculated using student t-test in (FIGS. 5A, 5B, 5C, and 5E). Two-side Wilcoxon Rank Sum Test method was applied in (FIG. 5G). * p<0.05; **p<0.01; ***p<0.001.

FIGS. 6A, 6B, 6C, 6D, and 6E show the synergistic effect of irisin with a-PD-1 antibody and its human relevance. FIG. 6A shows the schematic diagram of irisin and a-PD-1 antibody treatment plan. FIG. 6B shows the MC38 tumor growth curve of mice group treated with PBS, «-PD-1, irisin, and a-PD-1+ irisin. FIG. 6C shows the individual tumor growth curve in each treatment group. FIG. 6D show the quantification of each gene expression in CR (complete response)/PR (partial response) and SD (stable diseases)/PD (progressive diseases) groups. FIG. 6E shows the correlation of indicated gene expression with diseases free survival rate. Data are presented as mean±SEM. p values (FIG. 6B) were calculated using two-way ANOVA and student t-test was utilized for (FIGS. 6D and 6E). * p<0.05; **p<0.01; ***p<0.001.

FIGS. 7A, 7B, 7C, 7D, and 7E show that irisin treatment did not affect in vitro tumor cell growth but reduced Treg accumulation in vivo. FIG. 7A shows that cancer cells treated with increasing concentrations of irisin for 24 hours and viability assessed by Wst-1 assay. FIGS. 7B, 7C, 7D, and 7E show the tumor infiltrating lymphocytes were collected from day 15 MC38 tumors (treated with PBS or irisin) and stained using high-dimensional flow cytometry panel to assess immune cell population dynamics. CD45⁺ immune cells were gated using web-based software OMIQ. opt-sne dimension reduction and FlowSOM clustering identified 20 unique clusters with various expression patterns. FIG. 7B shows the opt-sne dimension reduction of CD45⁺ cells from PBS vs irisin-treated tumors. FIG. 7C shows the heatmaps of expression patterns from clusters in FIG. 7B. FIG. 7D shows the marker expression patterns and overlayed on UMAP space. FIG. 7E shows the edgeR analysis identified clusters with significantly altered frequencies in PBS vs. irisin treated tumors.

FIGS. 8A, 8B, 8C, 8D, 8E, and 8F show that irisin treatment did not alter immune cell population dynamics in secondary lymphoid organs. Tumor-draining lymph nodes (FIGS. 8A, 8B, and 8C) and spleens (FIGS. 8D, 8E, and 8F) were collected from implanted mice treated with PBS or irisin and stained using high-dimensional flow cytometry panel to assess immune cell population dynamics. CD45⁺ immune cells were gated using web-based software OMIQ. opt-sne dimension reduction and FlowSOM clustering identified unique clusters with various expression patterns. FIG. 8A shows the opt-sne dimension reduction showing 13 unique clusters comprising CD45⁺ cells from draining lymph nodes. FIG. 8B shows the heatmaps of expression patterns from clusters in FIG. 8A. FIG. 8C shows the edgeR analysis found no clusters with significantly altered frequencies.

FIG. 8D shows the opt-sne dimension reduction showing 15 unique clusters comprising CD45⁺cells from spleen. FIG. 8E shows the heatmaps of expression patterns from clusters in FIG. 8D. FIG. 8F edgeR analysis found no clusters with significantly altered frequencies.

FIGS. 9A, 9B, 9C, and 9D show that irisin treatment reduced suppressive phenotype of Tregs in early time point. Tumor-infiltrating lymphocytes were collected from day 10 MC38 tumors (treated with PBS or irisin) and stained using high-dimensional flow cytometry panel to assess immune cell population dynamics. CD4⁺ T cells were gated using web-based software OMIQ. UMAP dimension reduction and FlowSOM clustering identified 18 unique clusters with various expression patterns. FIG. 9A shows the UMAP of CD4⁺ T cells, combined. FIG. 9B shows the heatmap of expression patterns from clusters. FIG. 9C shows the contour plots of PBS vs irisin CD4⁺ T cells, with arrows indicating changes to cluster frequencies. FIG. 9D shows the edgeR analysis identified clusters with significantly altered frequencies in PBS vs. irisin-treated tumors. Clusters to the right of graph are upregulated, and those to the left are downregulated.

FIGS. 10A, 10B, 10C, and 10D show that irisin treatment resulted in retention of a progenitor exhausted CD8⁺ T cell subset and induction of a less-exhaustion related phenotype. Tumor-infiltrating lymphocytes were collected from day 10 MC38 tumors (treated with PBS or irisin) and stained using high-dimensional flow cytometry panel to assess immune cell population dynamics. CD8+ were gated using web-based software OMIQ. UMAP dimension reduction and FlowSOM clustering identified 16 unique clusters with various expression patterns. FIG. 10A shows the UMAP of CD8⁺ T cells, combined. FIG. 10B shows the heatmap of expression patterns from clusters. FIG. 10C shows the contour plots of PBS vs irisin CD8⁺ T cells, with arrows indicating changes to cluster frequencies. FIG. 10D shows the edgeR analysis identified clusters with significantly altered frequencies in PBS vs. irisin-treated tumors. Clusters to the right of graph are upregulated, and those to the left are downregulated.

FIGS. 11A and 11B show that integrin av and β5 are co-expressed in myeloid and fibroblast populations. FIG. 11A shows the publicly available data set from mouse melanoma samples was re-analyzed for cellular subsets and projected into UMAP space with clustering information. FIG. 11B shows the integrin av and β5 expression patterns (single or double positive) visualized in UMAP space.

FIGS. 12A, 12B, 12C, and 12D show that integrin av and β5 was co-expressed in myeloid and fibroblast population. FIGS. 12A and 12C shows the spatial transcriptomics data generated using patient colon cancer sample was analyzed. Representative H&E staining slide and spatial data visualizing cells both expressing integrin av and β5 (DP) and cells do not express either marker (Non-DP) was marked in FIG. 12A. TGF-β gene signature was applied to both categories and two-side Wilcoxon Rank Sum Test was utilized to evaluate statistical significance (FIGS. 12B and 12D). ***p<0.001.

FIGS. 13A and 13B show that irisin showed synergistic effect with a-PD-1 blockade therapy. FIG. 13A shows the survival curve representing immunotherapy response rate related to FIGS. 6 A, 6B, and 6C was plotted. FIG. 13B shows the tumor growth curve of MB49 tumor model where irisin and α-PD-1 blockade is combined. * p<0.05; **p<0.01; ***p<0.001.

### DETAILED DESCRIPTION

The following description of the disclosure is provided as an enabling teaching of the disclosure in its best, currently known embodiment(s). To this end, those skilled in the relevant art will recognize and appreciate that many changes can be made to the various embodiments of the invention described herein, while still obtaining the beneficial results of the present disclosure. It will also be apparent that some of the desired benefits of the present disclosure can be obtained by selecting some of the features of the present disclosure without utilizing other features. Accordingly, those who work in the art will recognize that many modifications and adaptations to the present disclosure are possible and can even be desirable in certain circumstances and are a part of the present disclosure. Thus, the following description is provided as illustrative of the principles of the present disclosure and not in limitation thereof.

Reference will now be made in detail to the embodiments of the invention, examples of which are illustrated in the drawings and the examples. This invention may, however, be embodied in many different forms and should not be construed as limited to the embodiments set forth herein.

### Terminology

Unless defined otherwise, all technical and scientific terms used herein have the same meaning as commonly understood to one of ordinary skill in the art to which this disclosure belongs. The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments and are also disclosed. As used in this disclosure and in the appended claims, the singular forms “a”, “an”, “the”, include plural referents unless the context clearly dictates otherwise.

The following definitions are provided for the full understanding of terms used in this specification.

The terms “about” and “approximately” are defined as being “close to” as understood by one of ordinary skill in the art. In one non-limiting embodiment the terms are defined to be within 10%. In another non-limiting embodiment, the terms are defined to be within 5%. In still another non-limiting embodiment, the terms are defined to be within 1%.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10”as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

As used herein, the terms “may,” “optionally,” and “may optionally” are used interchangeably and are meant to include cases in which the condition occurs as well as cases in which the condition does not occur. Thus, for example, the statement that a formulation “may include an excipient” is meant to include cases in which the formulation includes an excipient as well as cases in which the formulation does not include an excipient.

“Composition” refers to any agent that has a beneficial biological effect. Beneficial biological effects include both therapeutic effects, e.g., treatment of a disorder or other undesirable physiological condition, and prophylactic effects, e.g., prevention of a disorder or other undesirable physiological condition. The terms also encompass pharmaceutically acceptable, pharmacologically active derivatives of beneficial agents specifically mentioned herein, including, but not limited to, a vector, polynucleotide, cells, salts, esters, amides, proagents, active metabolites, isomers, fragments, analogs, and the like. When the term “composition” is used, then, or when a particular composition is specifically identified, it is to be understood that the term includes the composition per se as well as pharmaceutically acceptable, pharmacologically active vector, polynucleotide, salts, esters, amides, proagents, conjugates, active metabolites, isomers, fragments, analogs, etc.

An “increase” can refer to any change that results in a greater amount of a symptom, disease, composition, condition, or activity. An increase can be any individual, median, or average increase in a condition, symptom, activity, composition in a statistically significant amount. Thus, the increase can be a 1, 2, 3, 4, 5, 6, 7, 8, 9, 10, 15, 20, 25, 30, 35, 40, 45, 50, 55, 60, 65, 70, 75, 80, 85, 90, 95, or 100% increase so long as the increase is statistically significant.

A “decrease” can refer to any change that results in a smaller amount of a symptom, disease, composition, condition, or activity. A substance is also understood to decrease the genetic output of a gene when the genetic output of the gene product with the substance is less relative to the output of the gene product without the substance. Also, for example, a decrease can be a change in the symptoms of a disorder such that the symptoms are less than previously observed. A decrease can be any individual, median, or average decrease in a condition, symptom, activity, composition in a statistically significant amount. Thus, the decrease can be a 1, 2, 3, 4, 5, 6, 7, 8, 9, 10, 15, 20, 25, 30, 35, 40, 45, 50, 55, 60, 65, 70, 75, 80, 85, 90, 95, or 100% decrease so long as the decrease is statistically significant.

“Inhibit,” “inhibiting,” and “inhibition” mean to decrease an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

“Inhibitors” or “antagonist” of expression or of activity are used to refer to inhibitory molecules, respectively, identified using in vitro and in vivo assays for expression or activity of a described target protein, e.g., ligands, antagonists, and their homologs and mimetics. Inhibitors are agents that, e.g., inhibit expression or bind to, partially or totally block stimulation or activity, decrease, prevent, delay activation, inactivate, desensitize, or down regulate the activity of the described target protein, e.g., antagonists. Control samples (untreated with inhibitors) are assigned a relative activity value of 100%. Inhibition of a described target protein is achieved when the activity value relative to the control is about 80%, optionally 50% or 25, 10%, 5%, or 1% or less.

By “reduce” or other forms of the word, such as “reducing” or “reduction,” means lowering of an event or characteristic (e.g., tumor growth). It is understood that this is typically in relation to some standard or expected value, in other words it is relative, but that it is not always necessary for the standard or relative value to be referred to. For example, “reduces tumor growth” means reducing the rate of growth of a tumor relative to a standard or a control.

By “prevent” or other forms of the word, such as “preventing” or “prevention,” is meant to stop a particular event or characteristic, to stabilize or delay the development or progression of a particular event or characteristic, or to minimize the chances that a particular event or characteristic will occur. Prevent does not require comparison to a control as it is typically more absolute than, for example, reduce. As used herein, something could be reduced but not prevented, but something that is reduced could also be prevented. Likewise, something could be prevented but not reduced, but something that is prevented could also be reduced. It is understood that where reduce or prevent are used, unless specifically indicated otherwise, the use of the other word is also expressly disclosed.

The term “subject” refers to any individual who is the target of administration or treatment. The subject can be a vertebrate, for example, a mammal. In one aspect, the subject can be human, non-human primate, bovine, equine, porcine, canine, or feline. The subject can also be a guinea pig, rat, hamster, rabbit, mouse, or mole. Thus, the subject can be a human or veterinary patient. The term “patient” refers to a subject under the treatment of a clinician, e.g., physician.

The term “therapeutically effective” refers to the amount of the composition used is of sufficient quantity to ameliorate one or more causes or symptoms of a disease or disorder. Such amelioration only requires a reduction or alteration, not necessarily elimination.

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

“Comprising” is intended to mean that the compositions, methods, etc. include the recited elements, but do not exclude others. “Consisting essentially of” when used to define compositions and methods, shall mean including the recited elements, but excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions provided and/or claimed in this disclosure. Embodiments defined by each of these transition terms are within the scope of this disclosure.

As used herein, the term “buffer” refers to a solution consisting of a mixture of acid and its conjugate base, or vice versa. The solution is used as a means of keeping the pH at a nearly constant range to be used in a wide variety of chemical and biological applications.

The term “administer,” “administering”, or derivatives thereof refer to delivering a composition, substance, inhibitor, or medication to a subject or object by one or more the following routes: oral, topical, intravenous, subcutaneous, transcutaneous, transdermal, intramuscular, intra-joint, parenteral, intra-arteriole, intradermal, intraventricular, intracranial, intraperitoneal, intralesional, intranasal, rectal, vaginal, by inhalation or via an implanted reservoir. The term “parenteral” includes subcutaneous, intravenous, intramuscular, intra-articular, intra-synovial, intrasternal, intrathecal, intrahepatic, intralesional, and intracranial injections or infusion techniques.

The term “antibody” is used in the broadest sense, and specifically covers monoclonal antibodies (including full length monoclonal antibodies), polyclonal antibodies, and multispecific antibodies (e.g., bispecific antibodies). Antibodies (Abs) and immunoglobulins (Igs) are glycoproteins having the same structural characteristics. While antibodies exhibit binding specificity to a specific target, immunoglobulins include both antibodies and other antibody-like molecules which lack target specificity. Native antibodies and immunoglobulins are usually heterotetrameric glycoproteins of about 150,000 daltons, composed of two identical light (L) chains and two identical heavy (H) chains. Each heavy chain has at one end a variable domain (VH) followed by a number of constant domains. Each light chain has a variable domain at one end (VL) and a constant domain at its other end.

The term “cancer” is used to address any neoplastic disease, and is not limited to epithelial neoplasms (surface and glandular cancers; such a squamous cancers or adenomas)). It is used here to describe both solid tumors and hematologic malignancies, including epithelial (surface and glandular) cancers, soft tissue and bone sarcomas, angiomas, mesothelioma, melanoma, lymphomas, leukemias and myeloma.

A “T cell” refers to a type of lymphocyte that is one of the most important white blood cells of the immune system. T cells can be distinguished from other lymphocytes by the presence of a T-cell receptor (TCR) on their cell surface. The immune-mediated cell death function of T cells is carried by two major subtypes: CD8+ “killer” T cells and CD4⁺ “helper T cells.

The terms “treat,” “treating,” and grammatical variations thereof as used herein, include partially or completely delaying, alleviating, mitigating or reducing the intensity of one or more attendant symptoms of a disorder or condition and/or alleviating, mitigating or impeding one or more causes of a disorder or condition. Treatments according to the disclosure may be applied preventively, prophylactically, palliatively or remedially. Treatments are administered to a subject prior to onset (e.g., before obvious signs of tumor growth), during early onset (e.g., upon initial signs and symptoms of cancer), or after an established development of cancer.

A “therapeutically effective amount” of a drug necessary to achieve a therapeutic effect may vary according to factors such as the age, sex, and weight of the subject. Dosage regimens can be adjusted to provide the optimum therapeutic response. For example, several divided doses may be administered daily, or the dose may be proportionally reduced as indicated by the exigencies of the therapeutic situation.

“Effective amount” encompasses, without limitation, an amount that can ameliorate, reverse, mitigate, prevent, or diagnose a symptom or sign of a medical condition or disorder (e.g., cancer).

Unless dictated otherwise, explicitly or by context, an “effective amount” is not limited to a minimal amount sufficient to ameliorate a condition. The severity of a disease or disorder, as well as the ability of a treatment to prevent, treat, or mitigate, the disease or disorder can be measured, without implying any limitation, by a biomarker or by a clinical parameter.

“Pharmaceutically acceptable carrier” (sometimes referred to as a “carrier”) means a carrier or excipient that is useful in preparing a pharmaceutical or therapeutic composition that is generally safe and non-toxic and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents.

“Pharmaceutically acceptable” component can refer to a component that is not biologically or otherwise undesirable, i.e., the component may be incorporated into a pharmaceutical formulation of the invention and administered to a subject as described herein without causing significant undesirable biological effects or interacting in a deleterious manner with any of the other components of the formulation in which it is contained. When used in reference to administration to a human, the term generally implies the component has met the required standards of toxicological and manufacturing testing or that it is included on the Inactive Ingredient Guide prepared by the U.S. Food and Drug Administration.

As used herein, the term “carrier” encompasses any excipient, diluent, filler, salt, buffer, stabilizer, solubilizer, lipid, stabilizer, or other material well known in the art for use in pharmaceutical formulations. The choice of a carrier for use in a composition will depend upon the intended route of administration for the composition. The preparation of pharmaceutically acceptable carriers and formulations containing these materials is described in, e.g., Remington's Pharmaceutical Sciences, 21st Edition, ed. University of the Sciences in Philadelphia, Lippincott, Williams & Wilkins, Philadelphia, PA, 2005. Examples of physiologically acceptable carriers include saline, glycerol, DMSO, buffers such as phosphate buffers, citrate buffer, and buffers with other organic acids; antioxidants including ascorbic acid; low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN™ (ICI, Inc.; Bridgewater, New Jersey), polyethylene glycol (PEG), and PLURONICS™ (BASF; Florham Park, NJ). To provide for the administration of such dosages for the desired therapeutic treatment, compositions disclosed herein can advantageously comprise between about 0.1% and 99% by weight of the total of one or more of the subject compounds based on the weight of the total composition including carrier or diluent.

A “pharmaceutical composition” refers to at least one substance, molecule, or compound suitable for administering to a subject, wherein the composition further includes a pharmaceutical carrier. A non-limiting example include a therapeutic composition comprised of a protein and a sterile water-based solution.

### Methods

Irisin, a soluble myokine/adipokine primarily secreted by myocytes and/or adipocytes, was first discovered by P. Bostrom that demonstrated irisin concentrations in the blood following exercise. P. Boström also demonstrated that irisin provided improvements in obesity and glucose homeostasis in mice and humans. Bostrom et al is incorporated by reference for its teachings of the discovery and functions of irisin in humans and mice (Boström et al. “A PGC1-a-dependent myokine that drives brown-fat-like development of white fat and thermogenesis” Nature. Jan. 1, 2012; 481:463-469).

Studies have shown that exercise and exercise-induced hormones prevent cancer development, enhances efficacy of anti-tumor therapies, and prevents recurrence after treatment (Garcia-Chico et al. “Physiological Exercise and the Hallmarks of Breast Cancer: A Narrative Review”. Cancers 2023, 15 (1), 324; doi.org/10.3390/cancers15010324.; Ahmad Tali et al. “Significance of Physical Activity and Exercise in Cancer Patients: A Review on Exercise Oncology”. J Radiat Cancer Res. DOI: 10.4103/jrcr.jrcr_57_22.; Indorn et al. “Exercise and cancer: from ‘healthy’ to ‘therapeutic’?” Cancer Immunol Immunother. 21 Mar. 2017; 66, 667-671, doi.org/10.1007/s00262-017-1985-z). Exercise-induced hormones, such as insulin, cortisol, human growth hormone, testosterone, and epinephrine have also been shown to influence immune cell functions, specifically CD8⁺ T cells. It has been contemplated that irisin demonstrates similar regulatory influences over immune cell functions that provide improved treatment methods against diseases and disorder including, but not limited to cancer, chronic infections, and autoimmune diseases.

Presently, there remains limitations to current immunotherapies against cancer including, but not limited to development of resistance to current cancer immunotherapies, adverse reactions to immunotherapies, unpredictable efficacy, and high treatment costs. Thus, there is a need to develop improved methods of preventing, treating, decreasing, reducing, and/or ameliorating cancer. There is also a need to develop methods of enhancing, increasing, improving, and/or optimizing the efficacy of anti-tumor therapies.

Thus, the present disclosure provides methods of using irisin compositions to treat, prevent, decrease, reduce, and/or ameliorate cancer. The present disclosure also provides irisin as a biomarker for immunotherapy and cancer prognosis. In some embodiments, a subject's irisin levels are used as a biomarker for management of a cancer. In some embodiments, the subject's irisin levels are measured from a bodily samples (such as, for example blood, urine, serum, and tissue biopsy)

In one aspect, disclosed herein is a method of treating and/or preventing a cancer including, but not limited to colon carcinoma, melanoma, urothelial carcinoma, and lung cancer, in a subject, the method comprising administering a pharmaceutically effective amount of a composition comprising irisin and a pharmaceutically acceptable carrier, including, but not limited to an excipient, a diluent, a salt, a buffer, a stabilizer, a lipid, an emulsion, and a nanoparticle, wherein the composition prevents TGF-β activation in the subject relative to an untreated control.

In some embodiments, the method of treating and/or preventing a cancer comprises irisin targeting an integrin αvβ5 complex. In some embodiments, the method of treating and/or preventing a cancer comprises a composition that prevents release and activation of TGF-β in the subject relative to an untreated control. In some embodiments, the method of treating and/or preventing a cancer comprises a composition that reduces tumor growth in the subject relative to an untreated control. In some embodiments, the method of treating and/or preventing a cancer comprises a composition that causes accumulation of a functional T cell including, but not limited to a functional CD4⁺ T cell or a functional CD8⁺ T cell in the subject relative to an untreated control. In some embodiments, the method of treating and/or preventing a cancer comprises a composition that prevents accumulation of a regulatory T cell.

In some embodiments, the method of treating and/or preventing a cancer further comprises administering an immunotherapeutic agent or an immune checkpoint inhibitor including, but not limited to antibodies that block PD-1 (such as, for example, Nivolumab (BMS-936558 or MDX1106), pembrolizumab, cemiplimab, CT-011, MK-3475), PD-L1 (such as, for example, atezolizumab, avelumab, durvalumab, MDX-1105 (BMS-936559), MPDL3280A, or MSB0010718C), PD-L2 (such as, for example, rHIgM12B7), CTLA-4 (such as, for example, Ipilimumab (MDX-010), Tremelimumab (CP-675,206)), IDO, B7-H3 (such as, for example, MGA271, MGD009, omburtamab), B7-H4, B7-H3, T cell immunoreceptor with Ig and ITIM domains (TIGIT) (such as, for example BMS-986207, OMP-313M32, MK-7684, AB-154, ASP-8374, MTIG7192A, or PVSRIPO), CD96, B- and T-lymphocyte attenuator (BTLA), V-domain Ig suppressor of T cell activation (VISTA) (such as, for example, JNJ-61610588, CA-170), TIM3 (such as, for example, TSR-022, MBG453, Sym023, INCAGN2390, LY3321367, BMS-986258, SHR-1702, RO7121661), LAG-3 (such as, for example, BMS-986016, LAG525, MK-4280, REGN3767, TSR-033, BI754111, Sym022, FS118, MGD013, and Immutep) or a combination thereof.

In one aspect, disclosed herein is a method of preventing tumor growth factor β (TGF-β) activation in a tumor, the method comprising administering a composition comprising irisin and a pharmaceutically acceptable carrier, including, but not limited to an excipient, a diluent, a salt, a buffer, a stabilizer, a lipid, an emulsion, and a nanoparticle, to a subject with said tumor, and detecting a decreased amount of an activated TGF-β in the tumor relative to an amount of the activated TGF-β in a control tumor.

In some embodiments, the method of preventing TGF-β activation comprises irisin targets an integrin αvβ5 complex. In some embodiments, the method of preventing TGF-β activation comprises a composition that prevents release and activation of TGF-β. In some embodiments, the method of preventing TGF-β activation comprises a composition that reduces tumor growth in the subject relative to an untreated control. In some embodiments, the method of preventing TGF-β activation comprises a composition that causes accumulation of a functional T cell in the subject relative to an untreated control. In some embodiments, the method of preventing TGF-activation comprises a functional T cell including, but not limited to a CD4⁺ T cell or a CD8⁺ T cell. In some embodiments, the method of preventing TGF-β activation comprises a composition that prevents accumulations of a regulator T cell.

In some embodiments, the method of preventing TGF-β activation further comprises administering an immunotherapeutic agent or an immune checkpoint inhibitor including, but not limited to checkpoint inhibitor including, but not limited to antibodies that block PD-1 (such as, for example, Nivolumab (BMS-936558 or MDX1106), pembrolizumab, cemiplimab, CT-011, MK-3475), PD-L1 (such as, for example, atezolizumab, avelumab, durvalumab, MDX-1105 (BMS-936559), MPDL3280A, or MSB0010718C), PD-L2 (such as, for example, rHIgM12B7), CTLA-4 (such as, for example, Ipilimumab (MDX-010), Tremelimumab (CP-675,206)), IDO, B7-H3 (such as, for example, MGA271, MGD009, omburtamab), B7-H4, B7-H3, T cell immunoreceptor with Ig and ITIM domains (TIGIT) (such as, for example BMS-986207, OMP-313M32, MK-7684, AB-154, ASP-8374, MTIG7192A, or PVSRIPO), CD96, B- and T-lymphocyte attenuator (BTLA), V-domain Ig suppressor of T cell activation (VISTA) (such as, for example, JNJ-61610588, CA-170), TIM3 (such as, for example, TSR-022, MBG453, Sym023, INCAGN2390, LY3321367, BMS-986258, SHR-1702, RO7121661), LAG-3 (such as, for example, BMS-986016, LAG525, MK-4280, REGN3767, TSR-033, BI754111, Sym022, FS118, MGD013, and Immutep) or a combination thereof.

In some embodiments, the method of preventing TGF-β activation comprises treating, preventing, decreasing, and/or ameliorating a cancer including, but not limited to colon carcinoma, melanoma, urothelial carcinoma, and lung cancer. In some embodiments, the method of preventing TGF-β activation treats and/or prevents the cancer of any preceding aspect.

In one aspect, disclosed herein is a method of reducing tumor growth in a subject, the method comprising administering to a subject a pharmaceutically effective amount of a composition comprising irisin and a pharmaceutically acceptable carrier including, but not limited an excipient, a diluent, a salt, a buffer, a stabilizer, a lipid, an emulsion, and a nanoparticle, wherein the composition reduces tumor growth in the subject relative to an untreated control.

In some embodiments, the method of reducing tumor growth comprises targeting irisin to an integrin αvβ5 complex. In some embodiments, the method of reducing tumor growth comprises a composition that prevents release and activation of TGF-in the subject relative to an untreated control. In some embodiments, the method of reducing tumor growth comprises a composition that causes accumulation of a functional T cell, including but not limited to a functional CD4⁺ T cell or a functional CD8⁺ T cell, in the subject relative to an untreated control. In some embodiments, the method of reducing tumor growth comprises a composition that prevents accumulation of a regulator T cell.

In some embodiments, the method of reducing tumor growth treats and/or prevents a cancer including, but not limited to colon carcinoma, melanoma, urothelial carcinoma, and lung cancer.

In some embodiments, the method of reducing tumor growth further comprises administering an immunotherapeutic agent or an immune checkpoint inhibitor including, but not limited to checkpoint inhibitor including, but not limited to antibodies that block PD-1 (such as, for example, Nivolumab (BMS-936558 or MDX1106), pembrolizumab, cemiplimab, CT-011, MK-3475), PD-L1 (such as, for example, atezolizumab, avelumab, durvalumab, MDX-1105 (BMS-936559), MPDL3280A, or MSB0010718C), PD-L2 (such as, for example, rHIgM12B7), CTLA-4 (such as, for example, Ipilimumab (MDX-010), Tremelimumab (CP-675,206)), IDO, B7-H3 (such as, for example, MGA271, MGD009, omburtamab), B7-H4, B7-H3, T cell immunoreceptor with Ig and ITIM domains (TIGIT) (such as, for example BMS-986207, OMP-313M32, MK-7684, AB-154, ASP-8374, MTIG7192A, or PVSRIPO), CD96, B- and T-lymphocyte attenuator (BTLA), V-domain Ig suppressor of T cell activation (VISTA) (such as, for example, JNJ-61610588, CA-170), TIM3 (such as, for example, TSR-022, MBG453, Sym023, INCAGN2390, LY3321367, BMS-986258, SHR-1702, RO7121661), LAG-3 (such as, for example, BMS-986016, LAG525, MK-4280, REGN3767, TSR-033, BI754111, Sym022, FS118, MGD013, and Immutep) or a combination thereof.

It has been contemplated that the pharmaceutical composition comprising irisin of any preceding aspect can be effective in treating, preventing, decreasing, reducing, and/or ameliorating diseases including, but not limited to a proliferative disease, a chronic infection, or an autoimmune disease. Exemplary proliferative diseases include, but are not limited to, tumors, benign neoplasms, pre-malignant neoplasms (carcinoma in situ), and malignant neoplasms (cancers).

In some embodiments, the cancer of any preceding aspect includes, but is not limited to acoustic neuroma, adenocarcinoma, adrenal gland cancer, anal cancer, angiosarcoma (e.g., lymphangiosarcoma, lymphangioendotheliosarcoma, hemangiosarcoma), appendix cancer, benign monoclonal gammopathy, biliary cancer (e.g., cholangiocarcinoma), bladder cancer, breast cancer (e.g., adenocarcinoma of the breast, papillary carcinoma of the breast, mammary cancer, medullary carcinoma of the breast), brain cancer (e.g., meningioma; glioma, e.g., astrocytoma, oligodendroglioma; medulloblastoma), bronchus cancer, carcinoid tumor, cervical cancer (e.g., cervical adenocarcinoma), choriocarcinoma, chordoma, craniopharyngioma, colorectal cancer (e.g., colon cancer, rectal cancer, colorectal adenocarcinoma), epithelial carcinoma, ependymoma, endotheliosarcoma (e.g., Kaposi's sarcoma, multiple idiopathic hemorrhagic sarcoma), endometrial cancer (e.g., uterine cancer, uterine sarcoma), esophageal cancer (e.g., adenocarcinoma of the esophagus, Barrett's adenocarinoma), Ewing's sarcoma, eye cancer (e.g., intraocular melanoma, retinoblastoma), familiar hypereosinophilia, gall bladder cancer, gastric cancer (e.g., stomach adenocarcinoma), gastrointestinal stromal tumor (GIST), head and neck cancer (e.g., head and neck squamous cell carcinoma, oral cancer (e.g., oral squamous cell carcinoma (OSCC), throat cancer (e.g., laryngeal cancer, pharyngeal cancer, nasopharyngeal cancer, oropharyngeal cancer)), hematopoietic cancers (e.g., leukemia such as acute lymphocytic leukemia (ALL) (e.g., B-cell ALL, T-cell ALL), acute myelocytic leukemia (AML) (e.g., B-cell AML, T-cell AML), chronic myelocytic leukemia (CML) (e.g., B-cell CML, T-cell CML), and chronic lymphocytic leukemia (CLL) (e.g., B-cell CLL, T-cell CLL); lymphoma such as Hodgkin lymphoma (HL) (e.g., B-cell HL, T-cell HL) and non-Hodgkin lymphoma (NHL) (e.g., B-cell NHL such as diffuse large cell lymphoma (DLCL) (e.g., diffuse large B-cell lymphoma (DLBCL)), follicular lymphoma, chronic lymphocytic leukemia/small lymphocytic lymphoma (CLL/SLL), mantle cell lymphoma (MCL), marginal zone B-cell lymphomas (e.g., mucosa-associated lymphoid tissue (MALT) lymphomas, nodal marginal zone B-cell lymphoma, splenic marginal zone B-cell lymphoma), primary mediastinal B-cell lymphoma, Burkitt lymphoma, lymphoplasmacytic lymphoma (i.e., “Waldenstrom's macroglobulinemia”), hairy cell leukemia (HCL), immunoblastic large cell lymphoma, precursor B-lymphoblastic lymphoma and primary central nervous system (CNS) lymphoma; and T-cell NHL such as precursor T-lymphoblastic lymphoma/leukemia, peripheral T-cell lymphoma (PTCL) (e.g., cutaneous T-cell lymphoma (CTCL) (e.g., mycosis fungiodes, Sezary syndrome), angioimmunoblastic T-cell lymphoma, extranodal natural killer T-cell lymphoma, enteropathy type T-cell lymphoma, subcutaneous panniculitis-like T-cell lymphoma, anaplastic large cell lymphoma); a mixture of one or more leukemia/lymphoma as described above; and multiple myeloma (MM)), heavy chain disease (e.g., alpha chain disease, gamma chain disease, mu chain disease), hemangioblastoma, inflammatory myofibroblastic tumors, immunocytic amyloidosis, kidney cancer (e.g., nephroblastoma a.k.a. Wilms' tumor, renal cell carcinoma), liver cancer (e.g., hepatocellular cancer (HCC), malignant hepatoma), lung cancer (e.g., bronchogenic carcinoma, small cell lung cancer (SCLC), non-small cell lung cancer (NSCLC), adenocarcinoma of the lung), leiomyosarcoma (LMS), mastocytosis (e.g., systemic mastocytosis), myelodysplastic syndrome (MDS), mesothelioma, myeloproliferative disorder (MPD) (e.g., polycythemia Vera (PV), essential thrombocytosis (ET), agnogenic myeloid metaplasia (AMM) a.k.a. myelofibrosis (MF), chronic idiopathic myelofibrosis, chronic myelocytic leukemia (CML), chronic neutrophilic leukemia (CNL), hypereosinophilic syndrome (HES)), neuroblastoma, neurofibroma (e.g., neurofibromatosis (NF) type 1 or type 2, schwannomatosis), neuroendocrine cancer (e.g., gastroenteropancreatic neuroendoctrine tumor (GEP-NET), carcinoid tumor), osteosarcoma, ovarian cancer (e.g., cystadenocarcinoma, ovarian embryonal carcinoma, ovarian adenocarcinoma), papillary adenocarcinoma, pancreatic cancer (e.g., pancreatic adenocarcinoma, intraductal papillary mucinous neoplasm (IPMN), Islet cell tumors), penile cancer (e.g., Paget's disease of the penis and scrotum), pinealoma, primitive neuroectodermal tumor (PNT), prostate cancer (e.g., prostate adenocarcinoma), rectal cancer, rhabdomyosarcoma, salivary gland cancer, skin cancer (e.g., squamous cell carcinoma (SCC), keratoacanthoma (KA), melanoma, basal cell carcinoma (BCC)), small bowel cancer (e.g., appendix cancer), soft tissue sarcoma (e.g., malignant fibrous histiocytoma (MFH), liposarcoma, malignant peripheral nerve sheath tumor (MPNST), chondrosarcoma, fibrosarcoma, myxosarcoma), sebaceous gland carcinoma, sweat gland carcinoma, synovioma, testicular cancer (e.g., seminoma, testicular embryonal carcinoma), thyroid cancer (e.g., papillary carcinoma of the thyroid, papillary thyroid carcinoma (PTC), medullary thyroid cancer), urethral cancer, vaginal cancer and vulvar cancer (e.g., Paget's disease of the vulva).

In some embodiments, the cancer of any preceding aspect is indicated to be treated by immune checkpoint inhibitors targeting a checkpoint protein including, but not limited to PD-1 PD-L1 CTLA4, and Lag3.

As used herein, a “chronic infection” refers to an infection characterized by the continued presence of an infectious pathogen including, but not limited to a virus or bacterium, that causes prolonged or recurrent disease in a subject. In some embodiments, a subject with a chronic infection may symptomatic or may be asymptomatic. In some embodiments, the chronic infection includes, but is not limited to influenza (including, but not limited to human, bovine, avian, porcine, and simian strains of influenza), measles, acquired immune deficiency syndrome/human immunodeficiency virus (AIDS/HIV), anthrax, botulism, cholera, campylobacter infections, chickenpox, chlamydia infections, cryptosporidosis, dengue fever, diphtheria, hemorrhagic fevers, Escherichia coli (E. coli) infections, ehrlichiosis, gonorrhea, hand-foot-mouth disease, hepatitis A, hepatitis B, hepatitis C, legionellosis, leprosy, leptospirosis, listeriosis, malaria, meningitis, meningococcal disease, mumps, pertussis, polio, pneumococcal disease, paralytic shellfish poisoning, rabies, rocky mountain spotted fever, rubella, salmonella, shigellosis, small pox, syphilis, tetanus, trichinosis (trichinellosis), tuberculosis (TB), typhoid fever, typhus, west nile virus, yellow fever, yersiniosis, zika, endocarditis, myocarditis, eosinophilic myocarditis, pneumonia, bronchitis, emphysema, asbestosis, aspergilosis, severe acute respiratory syndrome (including, but not limited to SARS-COV-1 and SARS-COV-2), respiratory syncytial virus (RSV), middle eastern respiratory syndrome (MERS), cholecystitis, cholangitis, diarrhea, constipation, and clostridioides difficile infection.

As used herein, an “autoimmune disease or disorder” refers to a condition when a subject's immune system attacks and destroys healthy tissue because the immune system cannot distinguish between healthy tissue and harmful antigens/pathogens. As a result, the subject's body initiates a reactions of immune responses that destroys normal tissues. The result of an autoimmune disease/disorder comprises destruction of body tissue, abnormal growth of an organ, and/or changes in organ function. In some embodiments, the autoimmune disease/disorder includes, but it not limited to Addison disease, Celiac disease, Dermatomyositis, Graves disease, Hashimoto thyroiditis, inflammatory bowel diseases (such as, for example Chron's disease and ulcerative colitis), multiple sclerosis, myasthenia gravis, pernicious anemia, reactive arthritis, rheumatoid arthritis, systemic lupus erythematosus, and type I diabetes.

In some embodiments, the subject of any preceding aspect is a human.

The pharmaceutical composition and/or immunotherapeutic agent(s) (immune checkpoint inhibitor) may be administered in such amounts, time, and route deemed necessary in order to achieve the desired result. The exact amount of the pharmaceutical composition and/or immunotherapeutic agent(s) (immune checkpoint inhibitor) will vary from subject to subject, depending on the species, age, and general condition of the subject, the severity of the cancer, the particular pharmaceutical composition and/or immunotherapeutic agent(s) (immune checkpoint inhibitor), its mode of administration, its mode of activity, and the like. The pharmaceutical composition and/or immunotherapeutic agent(s) (immune checkpoint inhibitor) are preferably formulated in dosage unit form for ease of administration and uniformity of dosage. It will be understood, however, that the total daily usage of the pharmaceutical composition and/or immunotherapeutic agent(s) (immune checkpoint inhibitor) will be decided by the attending physician within the scope of sound medical judgment. The specific therapeutically effective dose level for any particular subject will depend upon a variety of factors including the cancer being treated and the severity of the cancer; the activity of the pharmaceutical composition and/or immunotherapeutic agent(s) (immune checkpoint inhibitor) employed; the specific pharmaceutical composition and/or immunotherapeutic agent(s) (immune checkpoint inhibitor) employed; the age, body weight, general health, sex and diet of the patient; the time of administration, route of administration, and rate of excretion of the specific pharmaceutical composition and/or immunotherapeutic agent(s) (immune checkpoint inhibitor) employed; the duration of the treatment; drugs used in combination or coincidental with the specific pharmaceutical composition and/or immunotherapeutic agent(s) (immune checkpoint inhibitor) employed; and like factors well known in the medical arts.

The pharmaceutical composition and/or immunotherapeutic agent(s) (immune checkpoint inhibitor) may be administered by any route. In some embodiments, the pharmaceutical composition and/or immunotherapeutic agent(s) (immune checkpoint inhibitor) are administered via a variety of routes, including oral, intravenous, intramuscular, intra-arterial, intramedullary, intrathecal, subcutaneous, intraventricular, transdermal, interdermal, rectal, intravaginal, intraperitoneal, topical (as by powders, ointments, creams, and/or drops), mucosal, nasal, buccal, enteral, sublingual; by intratracheal instillation, bronchial instillation, and/or inhalation; and/or as an oral spray, nasal spray, and/or aerosol. In general, the most appropriate route of administration will depend upon a variety of factors including the nature of the pharmaceutical composition and/or immunotherapeutic agent(s) (e.g., its stability in the environment of the subject's body), the condition of the subject (e.g., whether the subject is able to tolerate administration), etc.

The exact amount of the pharmaceutical composition and/or immunotherapeutic agent(s) (immune checkpoint inhibitor) required to achieve a therapeutically effective amount will vary from subject to subject, depending on species, age, and general condition of a subject, severity of the side effects, identity of the particular compound(s), mode of administration, and the like. The amount to be administered to, for example, a child or an adolescent can be determined by a medical practitioner or person skilled in the art and can be lower or the same as that administered to an adult.

In one aspect, disclosed herein is the pharmaceutical composition and/or immunotherapeutic agent(s) (immune checkpoint inhibitor) of any preceding aspect and a pharmaceutically acceptable carrier selected from an excipient, a diluent, a salt, a buffer, a stabilizer, a lipid, an emulsion, a nanoparticle, and a cream. One or more active agents (e.g. irisin and/or the immunotherapeutic agent (immune checkpoint inhibitor)) can be administered in the “native” form or, if desired in the form of salts, esters, amides, prodrugs, or a derivative that is pharmacologically suitable. Salts, esters, amides, prodrugs, and other derivatives of the active agents can be prepared using standards procedures known to those skilled in the art of synthetic organic chemistry and described, for example, by March (1992) Advanced Organic Chemistry; Reactions, Mechanisms, and Structure, 4th Ed. N.Y. Wiley-Interscience.

In some embodiments, the pharmaceutical composition and/or immunotherapeutic agent(s) (immune checkpoint inhibitor) can be prepared as a “concentrate”, e.g. in a storage container of a premeasure volume and/or a predetermined amount ready for dilution, or in a soluble capsule ready for addition to a specified volume of water, saline, alcohol, hydrogen peroxide, or other diluent.

In some embodiments, the pharmaceutical composition and/or immunotherapeutic agent(s) (immune checkpoint inhibitor) are administered 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or more times. In some embodiments, the pharmaceutical composition and/or immunotherapeutic agent(s) (immune checkpoint inhibitor) are administered daily. In some embodiments, the pharmaceutical composition and/or immunotherapeutic agent(s) (immune checkpoint inhibitor) are administered every day, every 2 days, every 3 days, every 4 days, every 5 days, every 6 days, every 7 days, or more. In some embodiments, the pharmaceutical composition and/or immunotherapeutic agent(s) (immune checkpoint inhibitor) are administered every week, every 2 weeks, every 3 weeks, every 4 weeks, or more. In some embodiments, the pharmaceutical composition and/or immunotherapeutic agent(s) (immune checkpoint inhibitor) are administered every month, every 2 months, every 3 months, every 4 months, every 5 months, every 6 months, every 7 months, every 8 months, every 9 months, every 10 months, every 11 months, every 12 months, or more. In some embodiments, the pharmaceutical composition and/or immunotherapeutic agent(s) (immune checkpoint inhibitor) are administered every year, every 2 years, every 3 years, every 4 years, every 5 years, or more.

A number of embodiments of the disclosure have been described. Nevertheless, it will be understood that various modifications may be made without departing from the spirit and scope of the invention. Accordingly, other embodiments are within the scope of the following claims.

By way of non-limiting illustration, examples of certain embodiments of the present disclosure are given below.

### Examples

The following examples are set forth below to illustrate the compositions, devices, methods, and results according to the disclosed subject matter. These examples are not intended to be inclusive of all aspects of the subject matter disclosed herein, but rather to illustrate representative methods and results. These examples are not intended to exclude equivalents and variations of the present invention which are apparent to one skilled in the art.

### Example 1: Exercise-Induced Hormone Irisin Potentiates Anti-Tumor Immunity Via Hindering Integrin Δv/TGF-β Axis in Tumor Microenvironment

Immunosuppressive cell populations within the tumor microenvironment (TME) negatively impact tumor control and are associated with worsened prognosis. One such population is regulatory T cells (Tregs), which are characterized by their immunoregulatory properties, and distinguished by expression of master transcription factor, forkhead box protein P3 (FOXP3). Under normal conditions, Tregs play a critical role in maintaining whole-body immune homeostasis. However, in patients with certain types of cancer, accumulation of Tregs in the TME is considered a poor prognostic factor. Tregs utilize multiple strategies to hinder tumor immunity. Cytotoxic T-lymphocyte associated protein-4 (CTLA-4) expression on Tregs can suppress function of antigen-presenting cells leading to CD8⁺ T cell dysfunction. Furthermore, Tregs compete with conventional T cells for critical cytokines including interleukin-2 (IL-2), which can cause suboptimal activation of non-Treg CD4⁺ and CD8⁺ T cells. Tregs also secrete immunosuppressive cytokines, such as transforming growth factor β (TGF-β) and IL-35, which interfere with tumor control. Due to their negative impact on outcomes in controlling tumor growth, multiple strategies to deplete or block differentiation of Tregs in the TME are under evaluation in the clinic.

TGF-β is a multifunctional immunosuppressive cytokine that controls effector function and differentiation of both adaptive and innate immune cell subsets. Major producers of TGF-β within the TME include tumor cells, Tregs, platelets, cancer-associated fibroblasts, and myeloid cells. TGF-β can directly suppress natural killer (NK) cell activity and stimulate expansion of Tregs, limiting the T cell-mediated anti-tumor immune response. The latent form of TGF-β (L-TGF-β) is generally present in the extracellular matrix complexed with latency-associated peptide (LAP), which covers its active domains to prevent receptor binding and signaling. Activation of L-TGF-β is largely mediated by integrin av complexes. Integrin av can pair with multiple β integrins (β-3, -5, -6, and -8); all of these complexes can bind and activate L-TGF-β. Binding of L-TGF-β to integrin αvβ3, -5, or -6 complexes induces an actin cytoskeletal force that releases active TGF-β from its inactive complex with LAP. Alternately, binding of L-TGF-β to integrin αvβ8 induces a conformational change that exposes the TGF-β active domain to initiate signaling, but without physical release of the active cytokine. While integrins have historically been studied in the context of cell-to-cell communication, intracellular signaling, and cell-to-extracellular matrix adhesion, their ability to activate L-TGF-β highlights their importance in cancer progression.

Exercise induces a systemic modulation of metabolism that provides numerous protective benefits against disease states, including cancer. Clinical studies have shown exercise enhances overall survival of cancer patients, reduces incidence, and limits side effects associated with treatment. In pre-clinical models, exercise augments the efficacy of anti-cancer therapies, reduces the rate and severity of adverse events, and prevents recurrence following treatment. However, the molecular mechanisms underlying these outcomes remain poorly understood. Benefits of exercise in the context of cancer include consumption of excessive energy by activated muscle cells and modulation of hormones. Exercise increases circulating hormone levels, including insulin, insulin-like growth factor-1, testosterone, thyroid-stimulating hormone (TSH), and irisin. Recent studies indicate the anti-tumor effects of exercise may be mediated by changes to the immune compartment, and exercise-induced hormones have been studied in this context. For instance, insulin receptor signaling, and growth hormone (somatotropin) pathway sustain CD8⁺ T cell proliferative capacity, and TSH affects T cell development. Irisin is a relatively recently identified exercised-induced hormone. Upon stimulation by exercise, muscle cells activate peroxisome proliferator-activated receptor gamma coactivator 1-alpha (PGC1-α) and upregulate expression of fibronectin type III domain containing 5 (FNDC5). The FNDC5 ectodomain is cleaved and secreted as irisin. Irisin was first identified as a metabolic regulator of fat tissue, where it converts white adipose tissue into brown/beige adipose tissue to increase energy expenditure, resulting in body weight reduction. Irisin also facilitates muscle regeneration and rescues synaptic plasticity in an Alzheimer's disease model. In the context of cancer, irisin can limit pancreatic cancer cell growth in vitro, and lower levels of irisin were detected in serum from breast cancer patients compared to healthy donors. Lower levels of irisin were also associated with increased spinal metastases in breast cancer patients. However, the role of irisin in anti-tumor immunity has not yet been defined.

Herein, irisin is reported to suppress tumor growth in multiple pre-clinical models through T cell-dependent mechanisms. Irisin treatment reduced accumulation of intra-tumoral Tregs at early and late time points, leading to less dysfunction in CD8⁺ T cells. To understand the underlying molecular mechanisms of these findings, a publicly available single-cell RNA-sequencing and spatial transcriptomics data sets were analyzed to evaluate the expression patterns of integrin av/β5 complex, a known binding partner of irisin. Both integrins were co-expressed in the myeloid cell compartment and irisin treatment was associated with reduced activation of L-TGF-β. As expected, anti-tumor effects of irisin were lost in mice with a myeloid cell-specific knockout of av integrin, implicating integrin av mediates the effect of irisin. Finally, combining irisin with α-PD-1 antibody overcomes tumor resistance to checkpoint blockade. Thus, a mechanism was identified by which the exercise-induced hormone irisin enhances CD8⁺ T cell mediated anti-tumor immunity by blocking both L-TGF-β activation and Treg accumulation in the TME.

### Results.

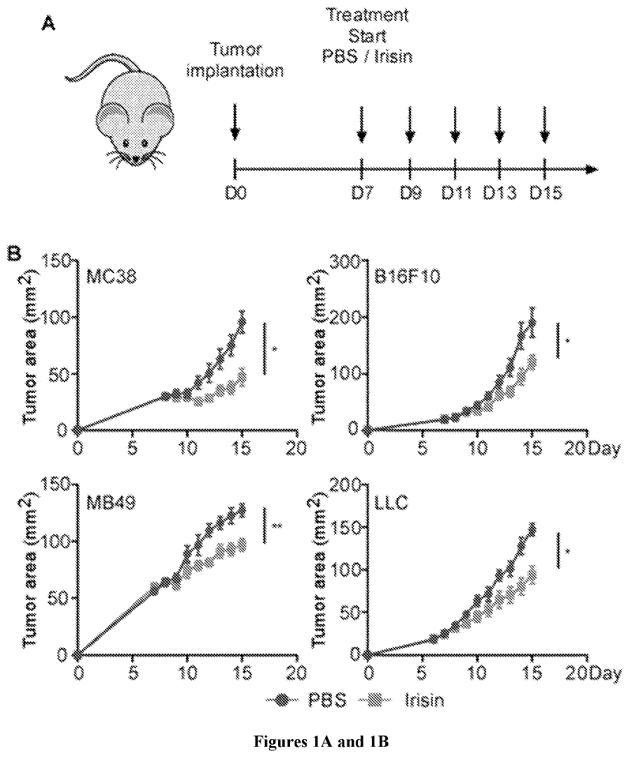

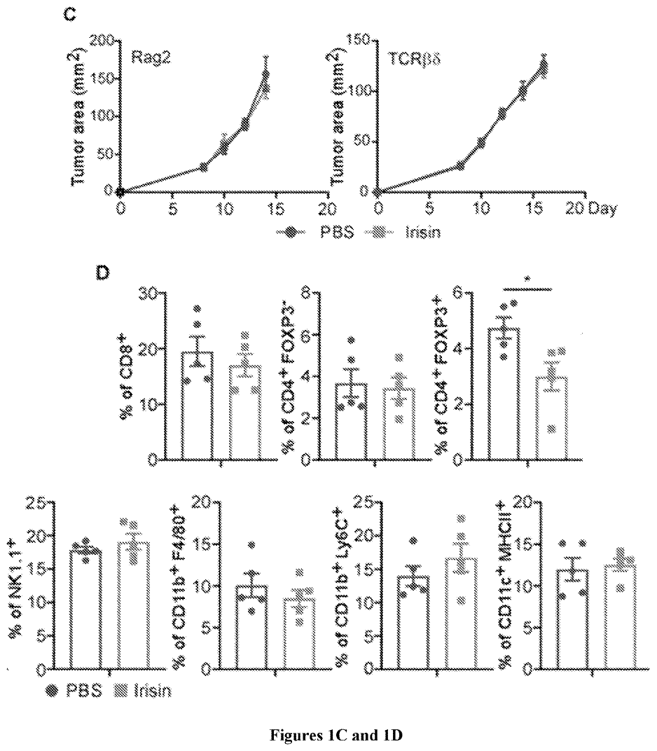

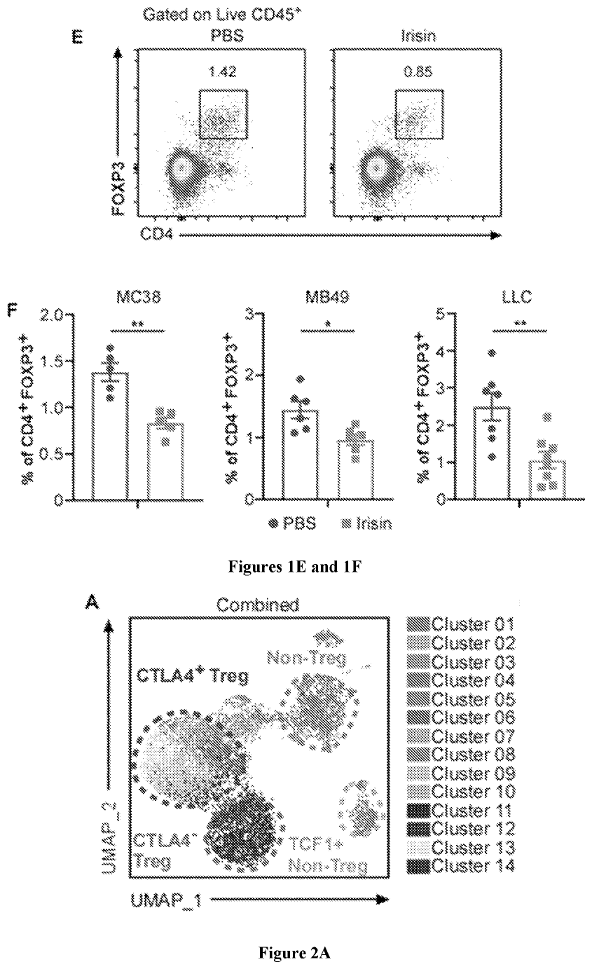

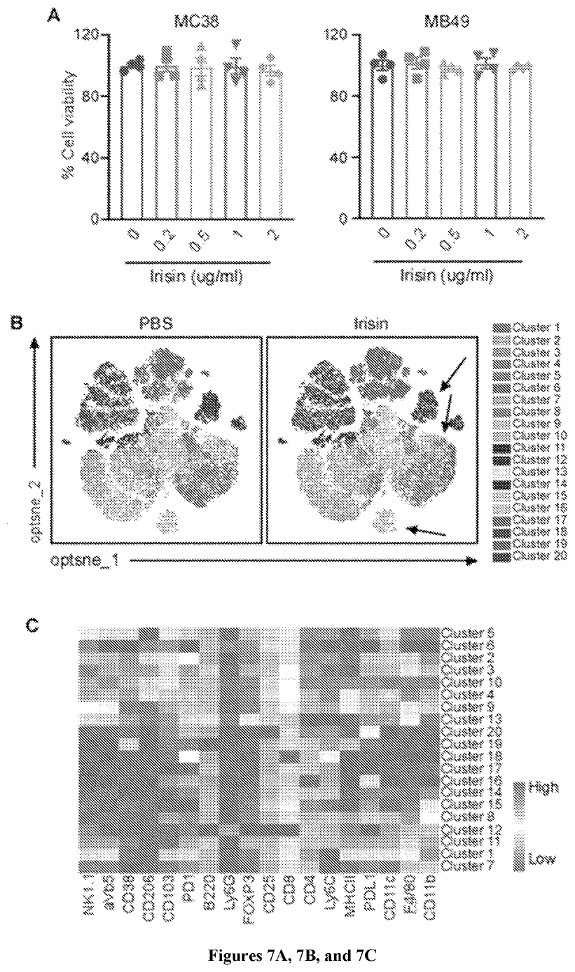

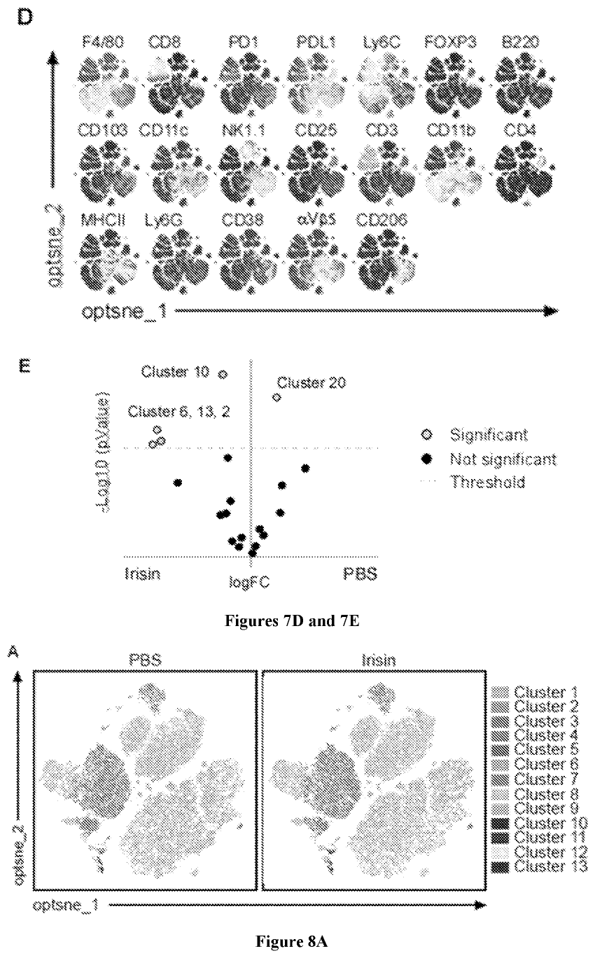

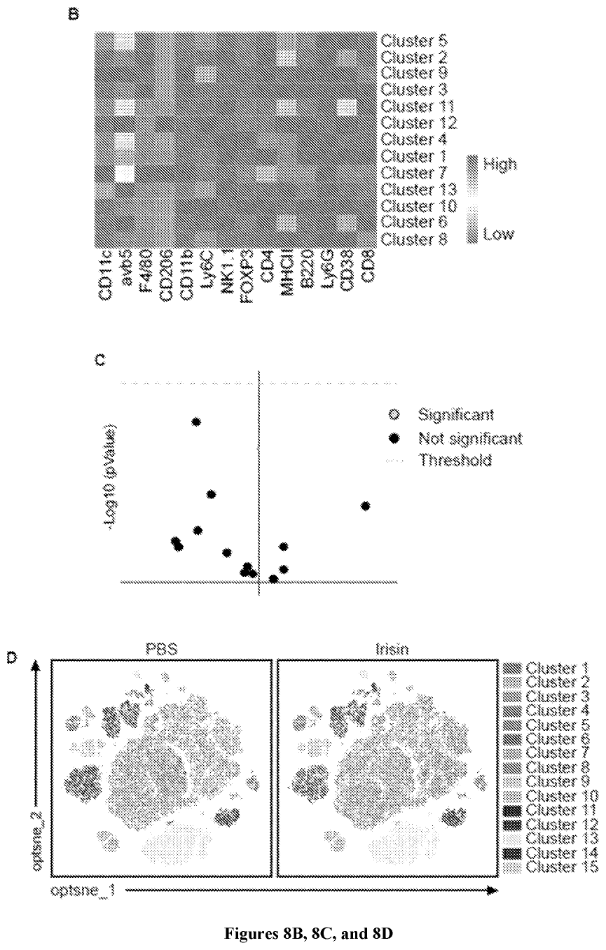

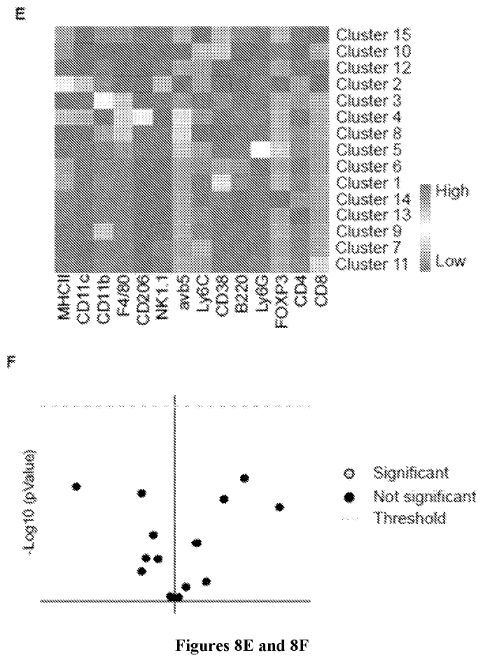

Irisin reduced tumor growth in multiple pre-clinical models through a T cell-dependent mechanism. Syngeneic subcutaneous tumor models were used to evaluate the impact of irisin treatment on tumor growth, including MC38 (colon carcinoma), β16F10 (melanoma), MB49 (urothelial carcinoma), and LLC (Lewis lung carcinoma). Irisin treatment started seven days after tumor implant in C57BL/6J mice and continued every two days for five doses (FIG. 1A). Irisin significantly reduced tumor growth in all models studied (FIG. 1B), but therapeutic efficacy was abolished when tumors were implanted in immune-deficient RAG2 knockout (lacking B and T cells) and T cell receptor (TCR) βδ knockout (lacking αβ and γδ TCR) mice (FIG. 1C). This showed a T cell-dependent mechanism was modulating the anti-tumoral effects of irisin. To determine whether irisin directly altered tumor cell growth, MC38 and MB49 cell lines were treated with irisin in vitro. Irisin did not impact tumor cell growth kinetics in vitro (FIG. 7A); therefore, its ability to modulate immune cells within the TME was focused upon. Using the MC38 model and treatment schematic outlined in FIG. 1A, day 10 tumors (early stage, before tumor volume significantly diverged between groups) were collected, isolated single cells, and stained them with a high-dimensional flow cytometry panel containing multiple lineage markers to assess immune cell population dynamics. Major cell populations were manually gated, and irisin treatment reduced intra-tumoral accumulation of Tregs (CD4⁺ FOXP3⁺; FIG. 1D). To further validate these findings, high-dimensional flow cytometry data was processed using the web-based software OMIQ. Live CD45⁺ immune cells were gated and opt-sne dimension reduction technique and FlowSOM clustering analysis was performed. Twenty clusters representing distinct immune cell populations were identified (FIG. 7B). Characteristics of each population was validated using clustered heatmap and individual marker expressions (FIGS. 7C and 7D). Decreased Treg frequency was confirmed with irisin treatment using this analysis (FIGS. 7B and 7E). Finally, day 15 (late-stage) tumors from MC38, MB49, and LLC models were analyzed and also showed reduced Treg accumulation within the TME (FIG. 1E-F). Of note, differences in population dynamics were not observed in secondary lymphoid organs, such as spleen and draining lymph nodes when similar analytical approaches were applied (FIG. 8).

Irisin reduced suppressive phenotype in intra-tumoral Tregs. Because irisin reduced intra-tumoral Treg accumulation, next this population was characterized using high-dimensional flow cytometry, which comprehensively defines T cell activation and exhaustion statuses. Day 15 MC38 tumors (after four doses of irisin or PBS treatment) were harvested, and high-dimensional flow cytometry was utilized. CD4⁺ T cells were gated in OMIQ and dimension reduction using uniform manifold approximation and projection (UMAP) and FlowSOM for clustering analysis was applied.

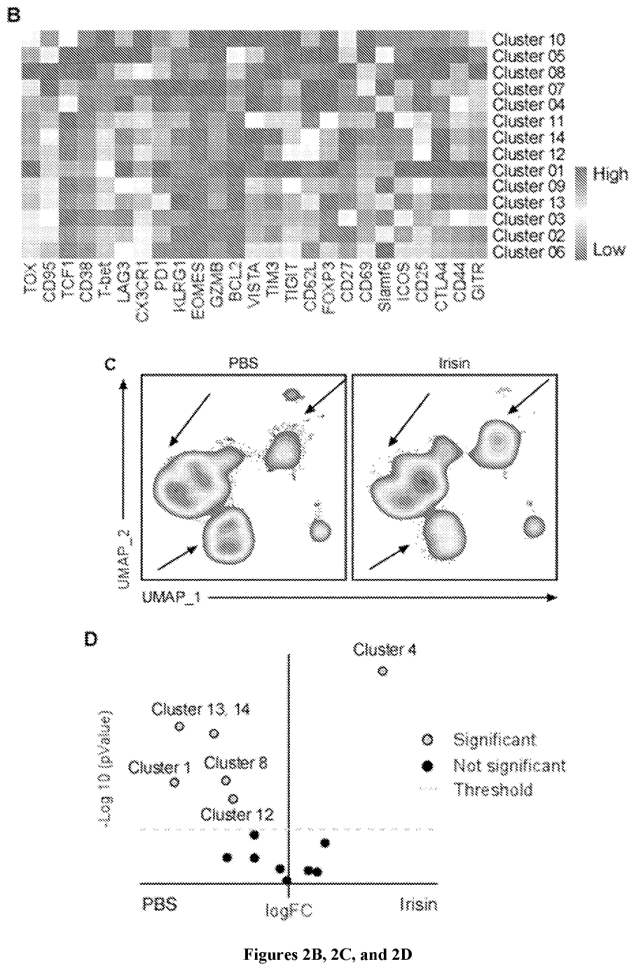

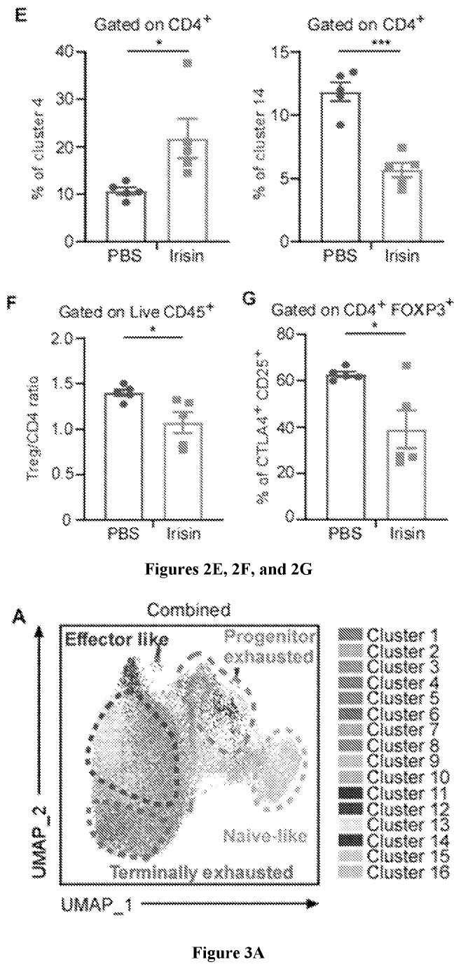

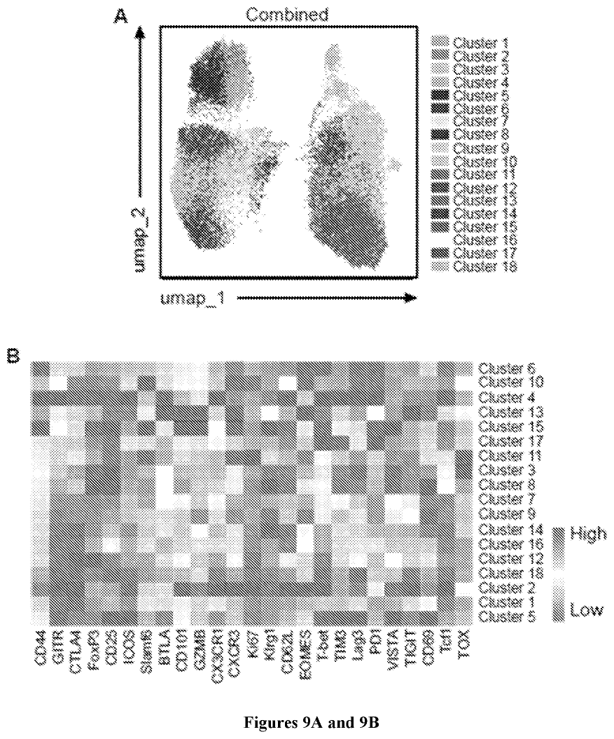

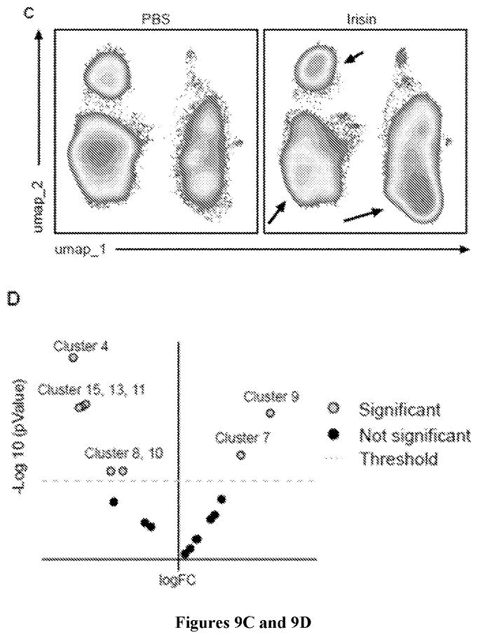

14 distinct clusters were found within the CD4⁺ T cell population with unique marker expression patterns, which were visualized using UMAP and heatmaps (FIG. 2A-B). The CD4⁺ T cells from control were compared to irisin-treated tumors using contour plots, and edgeR analysis was performed to depict statistically different clusters (FIG. 2C-D). Treg clusters 1 and 13 were significantly reduced following irisin treatment (top right arrow, FIG. 2C); these two clusters had the highest expression of CTLA4 (FIG. 2B), indicating a higher suppressive function. Other Treg Clusters 12 and 14 (bottom left arrow, FIG. 2C) were also decreased; these populations did not express CTLA4 but did express multiple other inhibitory receptors, including VISTA and TIM3 (FIG. 2B). Non-Treg Cluster 4 (top right arrow, FIG. 2C), which was characterized by low expression of FOXP3 and CD25, was increased following irisin treatment. Differences in population dynamics were verified by comparing specific cluster frequencies with different statistical approach (FIG. 2E). Using 2D flow cytometry plots, an overall change within the CD4⁺ T cell compartment was validated using a Treg/non-Treg CD4⁺ T cell ratio (FIG. 2F). The irisin treatment was confirmed to be associated with reduced suppressive function in Tregs by comparing the frequency of CTLA4⁺CD25⁺ cells (FIG. 2G). Finally, these results were also confirmed using samples from day 10 MC38 tumors (collected after two doses of irisin or PBS treatment). Even at early stage, irisin treatment is associated with reduced accumulation of Tregs and lower suppressive capacity (FIGS. 9A, 9B, 9C, and 9D).

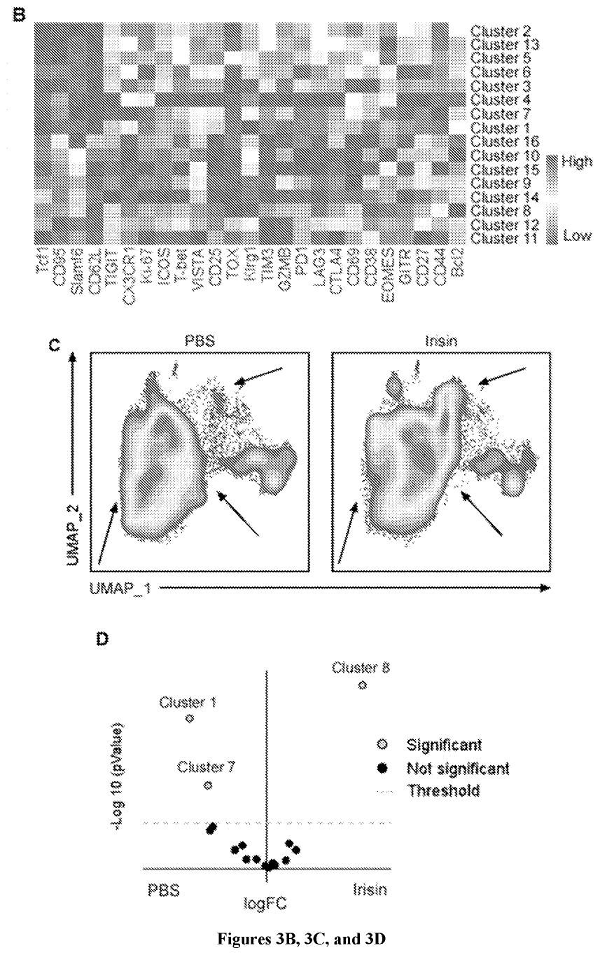

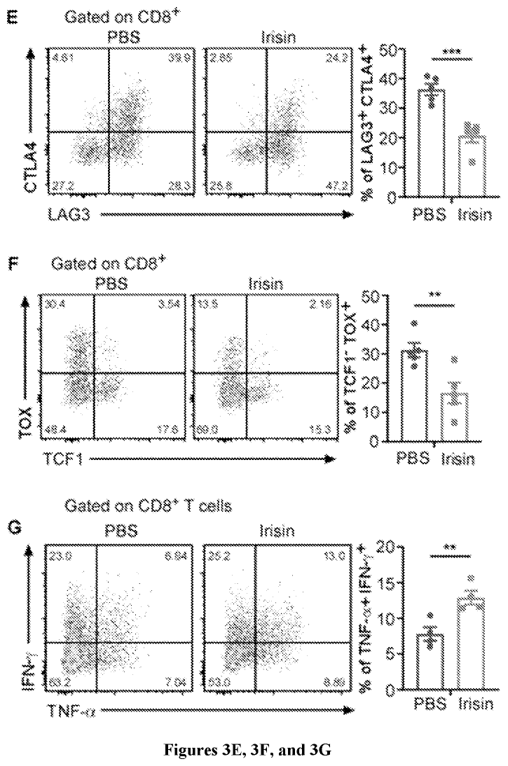

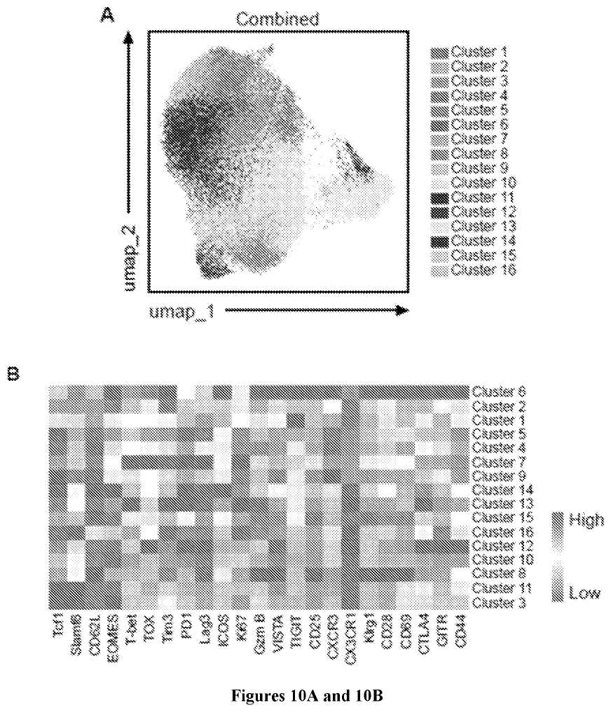

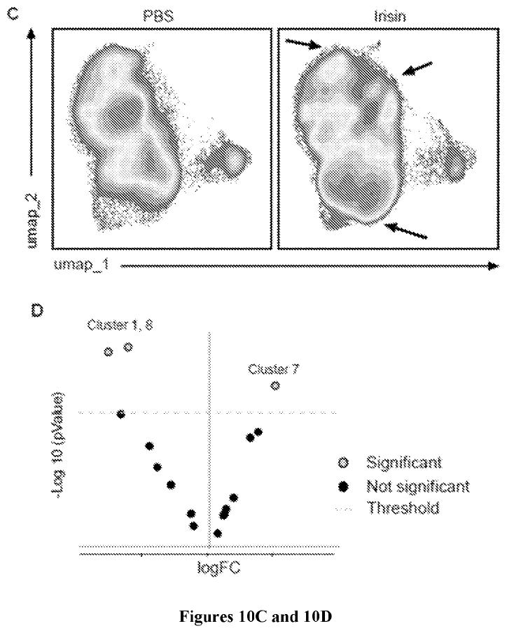

Irisin treatment prevents the formation of CD8⁺ T cells with dysfunctional signatures in the TME. Tregs within the TME play a key role in suppressing cytotoxic CD8⁺ T cell activity. Since irisin modulates Treg frequency and their suppressive capacity, irisin treatment was examined for associations with changes in CD8⁺ T cell phenotype using the high-dimensional flow cytometry analyses described above. CD8⁺ T cells were gated from day 15 MC38 tumors (after four doses of irisin or PBS treatment), and UMAP dimension reduction and FlowSOM clustering was applied. 16 distinct clusters were identified and further visualized their expression characteristics using a clustered heatmap (FIG. 3A-B). Contour plots of CD8⁺ T cells from PBS were compared to irisin-treated tumors and used edgeR analysis to detect significantly affected clusters (FIG. 3C-D). Irisin treatment reduced clusters 1 and 7 (bottom-left arrow, FIG. 3C), which are dysfunctional populations characterized by higher expression of inhibitory molecules. Supporting these findings, it was found that irisin treatment significantly reduced accumulation of dysfunctional LAG3+ CTLA4⁺CD8⁺ T cells intratumorally (FIG. 3E). Additionally, fewer TOX single-positive cells in irisin-treated samples were found, further indicating reduced accumulation of dysfunctional CD8⁺ T cells (FIG. 3F). Underscoring these findings, CD8⁺ T cells from irisin treated day 10 MC38 tumors (two doses of irisin) showed reduced accumulation of dysfunctional CD8⁺ T cells and retention of stem-like populations that can be further activated into effector CD8⁺ T cells (FIGS. 10A, 10B, 10C, and 10D). To test the cytokine production capacity of CD8⁺ T cells from PBS vs irisin treated tumors, cells were stimulated ex vivo and measured tumor necrosis factor alpha (TNF-α) and interferon gamma (IFN-γ) expression via flow cytometry. Consistent with previous results characterized by inhibitory and effector molecule expression patterns, CD8⁺ T cells from irisin treated tumors showed higher cytokine expression (FIG. 3G).

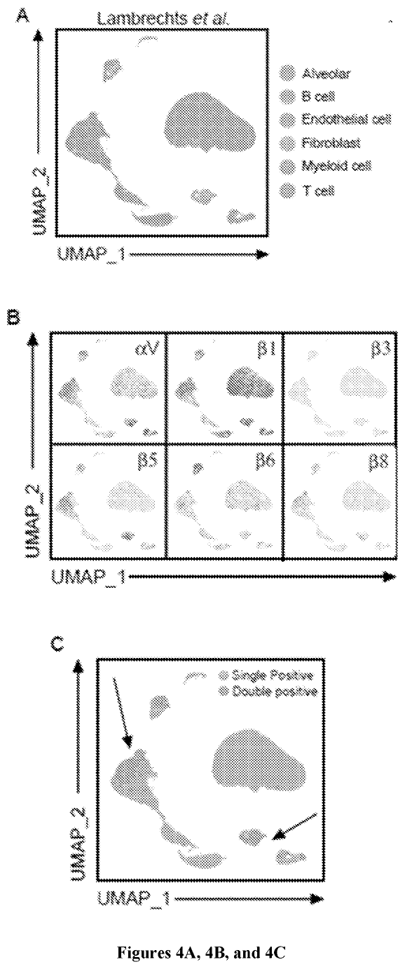

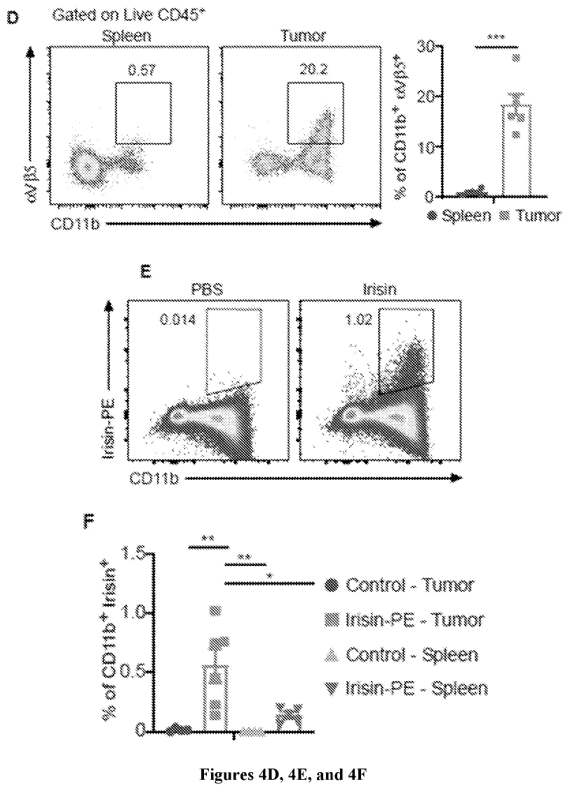

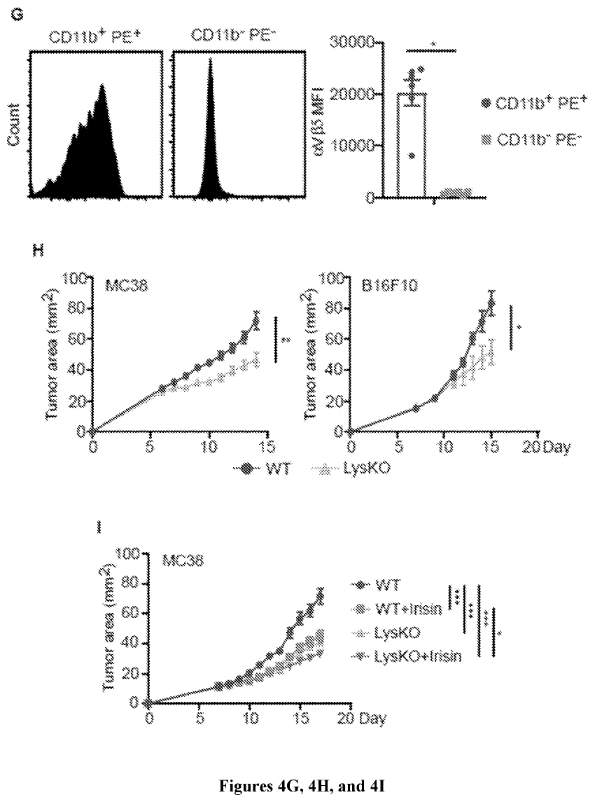

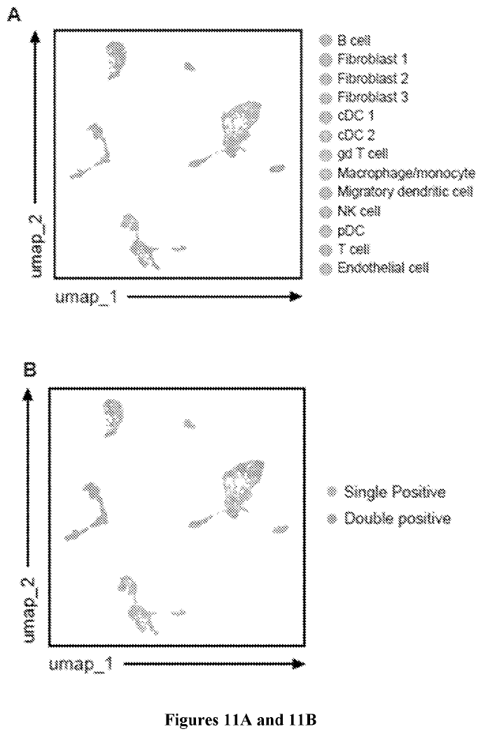

Integrin αvβ5 complex, a known target of irisin, is expressed by myeloid cells and mediates effect of irisin on tumor control. To investigate the molecular mechanisms by which irisin exerts its anti-tumor effects, the expression pattern of the integrin αvβ5 complex, a well-established binding partner of irisin, was first evaluated. A publicly available scRNA-seq dataset from human lung cancer samples was re-analyzed. First, cell clusters in UMAP space were annotated and then evaluated expression of integrin av and its β binding partners (FIG. 4A-B). Integrin av was specifically expressed in cancer-associated fibroblasts, endothelial cells, and myeloid cells. Myeloid cells and fibroblast populations expressed integrin β5. Integrin β1 was expressed by multiple cell populations, and β6 was expressed by alveolar cells and a subset of B cells. Integrins β3 and β8 showed minimal expression in this dataset (FIG. 4B). Interestingly, most cells expressing both integrin av and β5 were a subset of myeloid cells (FIG. 4C). This specific expression pattern was validated using a publicly available dataset generated from mouse tumor samples (FIGS. 11A and 11B). Next, flow cytometry was used to examine this expression pattern in immune cells from spleen and tumor from MC38 tumor bearing mice. Integrin αvβ5 was highly expressed on CD11b⁺ cells, indicating the myeloid cell compartment (FIG. 4D). To verify whether exogenously administered irisin can localize to the tumor and bind integrin αvβ5 therein, tumor-bearing mice were injected with fluorochrome-tagged irisin. Two hours post-injection, tumor and spleen was collected and assessed for irisin-PE and CD11b levels (FIG. 4E). A higher frequency of irisin-PE in tumor samples was detected, and these PE-positive cells co-expressed CD11b (FIG. 4F). Integrin αvβ5 expression was also assessed in the CD11b⁺ PE⁺ double positive vs CD11b PE-double negative populations. PE-positive cells had significantly higher integrin αvβ5 mean fluorescence intensity (MFI), showing that irisin binds to myeloid cells with higher integrin αvβ5 expression levels (FIG. 4G). To determine whether expression of integrin av in myeloid cells impacts tumor growth, a myeloid cell-specific knockout system was created. LysMcre mice were crossed with integrin av^{fl/fl} mice, whose Itgav gene is flanked by loxP sites to create Cre-negative/av^{fl/fl} (WT) mice and Cre positive/av^{fl/fl} (LysMKO) mice. Reduced tumor growth was observed in LysMKO in both MC38 and β16F10 tumor models (FIG. 4H). When MC38 tumor bearing WT and LysMKO mice were treated with irisin, irisin reduced tumor growth in the WT group, but this suppressive effect was largely abolished in LysMKO mice (FIG. 4I). These data show a critical role for integrin av in irisin mediated tumor control.

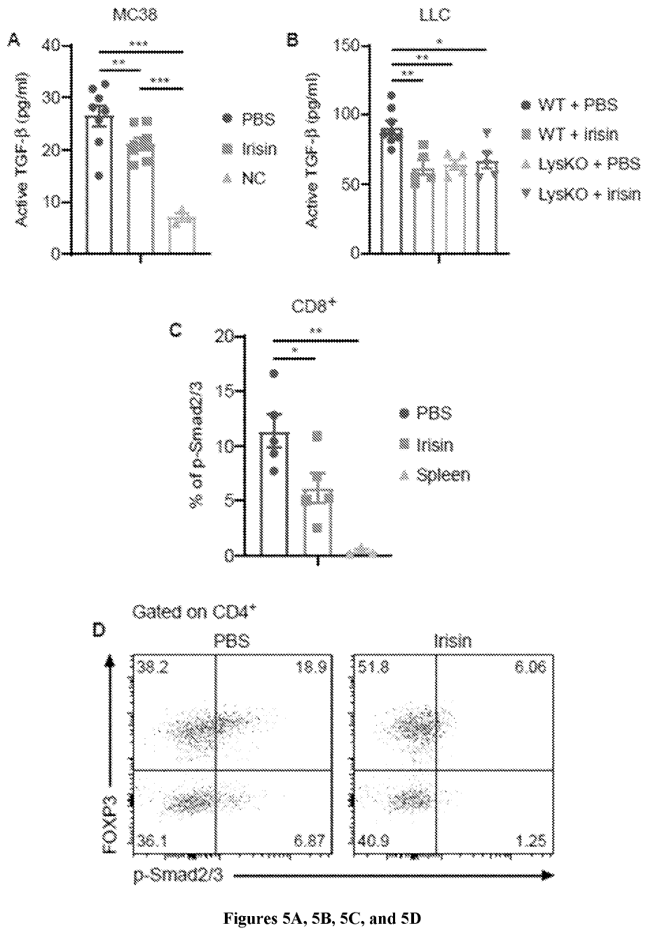

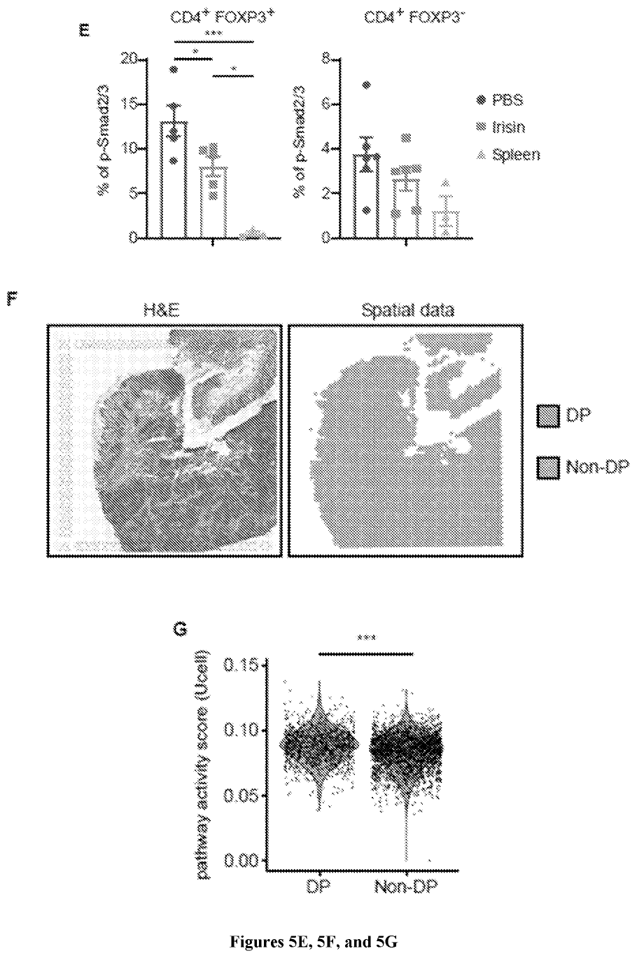

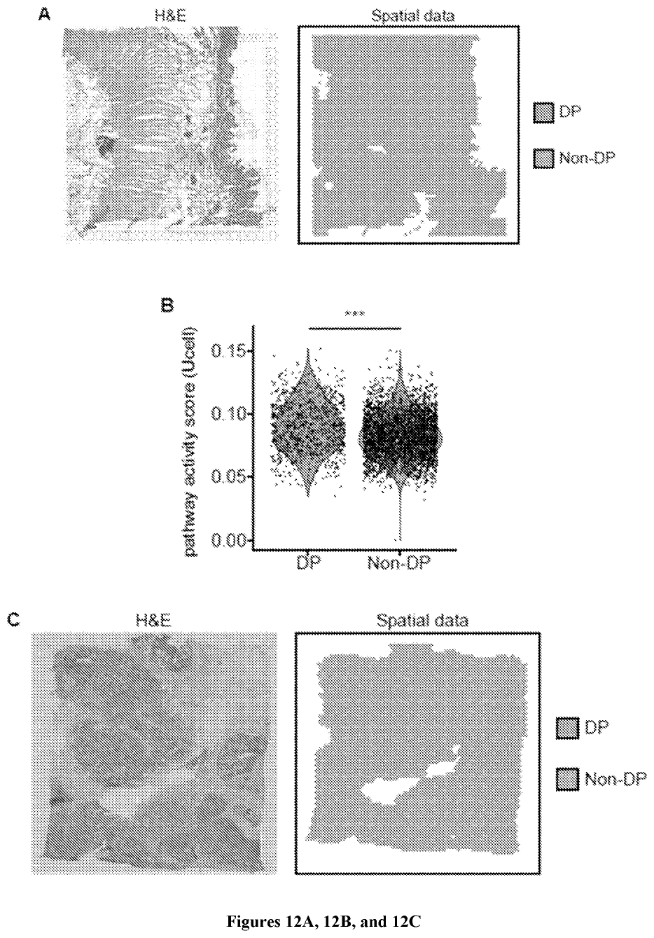

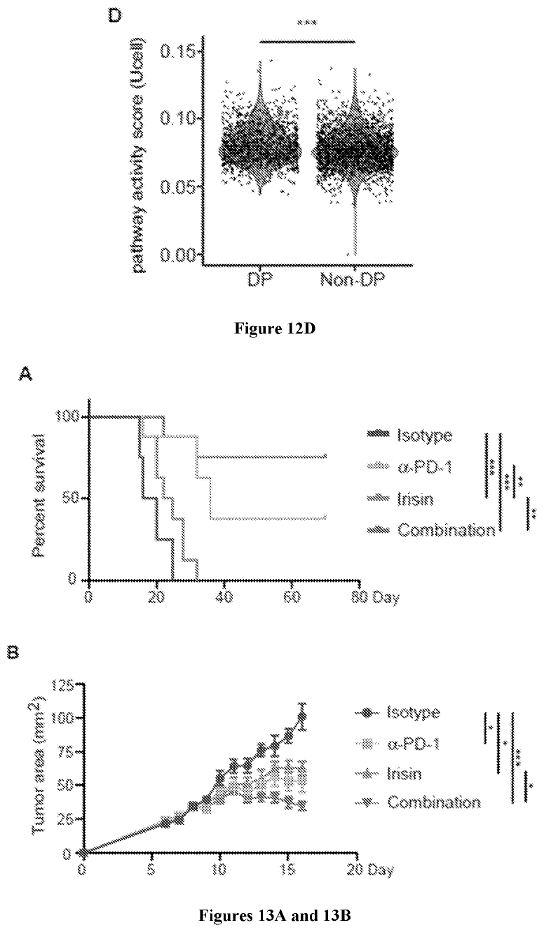

Irisin inhibited integrin αvβ5 mediated L-TGF-β activation. TGF-β is a multi-functional cytokine that exerts immunosuppressive pressures in the TME. It is generally presented as the latent TGF-β (L-TGF-β) complex, and its release has been studied in the context of both cancer progression and therapy. Integrin av complexes are main mediators of TGF-β release and activation, and TGF-β in the TME is a primary driver of Treg development and differentiation. Since less accumulation of Tregs were observed in tumors following irisin treatment (FIG. 1) and given the fact that integrin αvβ5 is known binding partner of irisin, the irisin treatment reduced activation of L-TGF-β indirectly by occupying the integrin αvβ5 complex. To evaluate this, myeloid cells were isolated from MC38 tumors and co-cultured with exogenous L-TGF-β+ irisin for 2 hours to test its activation level. Active TGF-β levels were assessed in the supernatant using an ELISA kit and found that irisin treated myeloid cells had reduced capacity to activate TGF-β (FIG. 5A). Next, isolated CD11b⁺ myeloid cell population from day 15 LLC tumors grown in WT and LysMKO mice were treated with irisin or control for 4 doses. Myeloid cells were incubated with L-TGF-β for two hours, and the media were collected to assess the levels of active TGF-β. As expected, cells from the PBS treated group led to the highest active TGF-β formation, whereas cells from LysMKO mice produced a lower amount of active TGF-β. In vivo irisin treatment was able to reduce active TGF-β formation, but a synergistic effect was not observed when irisin was treated in LysMKO mice, further suggesting involvement of integrin av in this process (FIG. 5B). To test whether less conversion of L-TGF-β within the TME caused biologically meaningful changes, phosphorylated SMAD2/3 levels were checked, which directly reflect TGF-β activity in CD8+ and CD4⁺ T cells. CD8⁺ T cells from day 14 MC38 tumors treated with irisin exhibited less p-SMAD2/3 levels assessed by flow cytometry, and spleen CD8⁺T cells showed minimal expression of p-SMAD2/3 (FIG. 5C). Next, changes in CD4⁺ T cell compartments were assessed and then utilized FOXP3 to compare the differences between non-Treg CD4⁺ T cells and Tregs (FIG. 5D). Irisin treatment significantly reduced p-SMAD2/3 positive Tregs, but non-Treg CD4⁺ T cells only showed trend (FIG. 5E). To further verify the relationship between the integrin αvβ5 complex and the TGF-β signaling pathway in the TME, a publicly available spatial transcriptomics dataset established with human colon cancer samples was used. First, cells expressing both integrin av and β5 and spatial spots expressing both integrins were identified and clustered as double positive (DP). Cells remaining at distal areas were clustered as non-DP (FIG. 5F). To address the relationship between TGF-β and integrin αvβ5, the TGF-β signaling pathway gene signature was downloaded from Gene Set Enrichment Analysis (GSEA) and applied to the spatial dataset. The pathway activity score was higher in the DP area confirming an association between TGF-β and integrin αvβ5 (FIG. 5G). This finding was validated using a colon cancer sample from a different patient and another dataset generated from a breast cancer patient (FIG. 12). These data show the critical role of irisin in blocking the TGF-β signaling pathway in the TME, leading to better tumor control mediated by T cells.

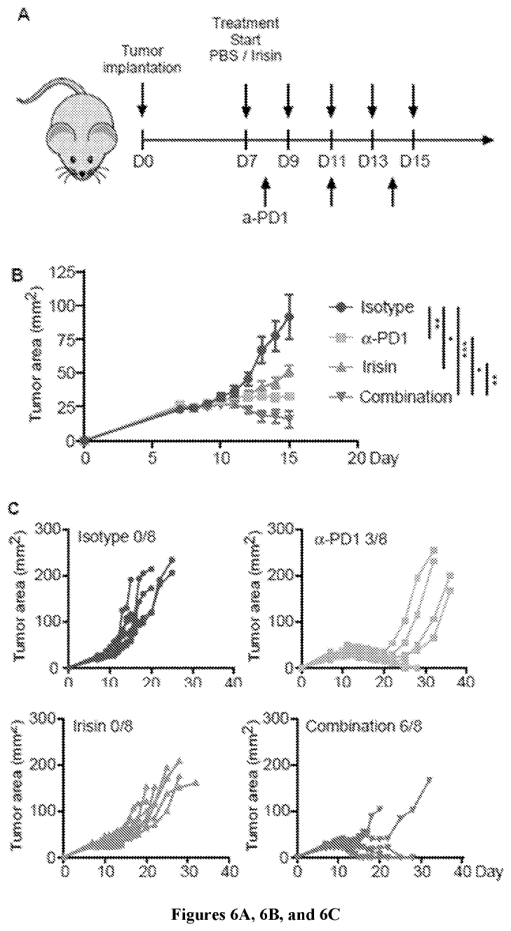

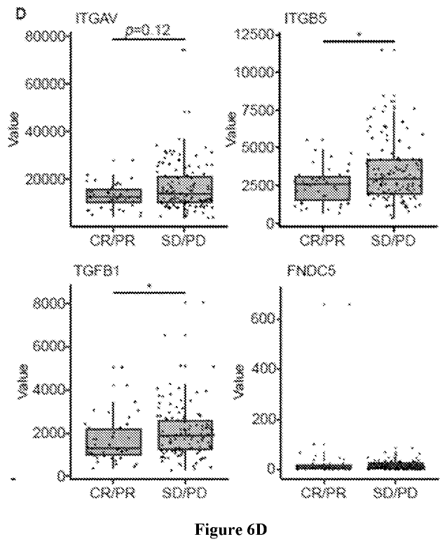

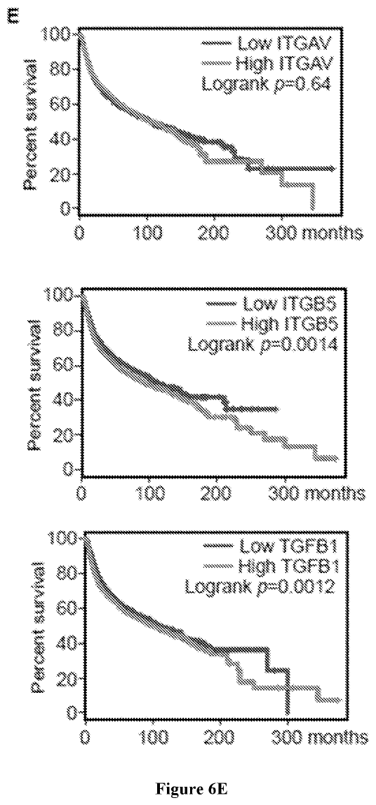

Irisin showed a synergistic effect with PD-1 blockade therapy. Immunotherapy that blocks the PD-1 and PD-L1 axis in the TME has shown outstanding promise in treating cancer patients. As irisin treatment affects the CD8⁺ T cell dysfunctional state, the combinatorial treatment of irisin with PD-1 blockade therapy was assessed. Irisin was injected per the previous treatment schedule, and a sub-optimal dose of α-PD-1 antibody (60 ug) injection was started on day 8 to assess therapeutic potential (FIG. 6A). The sub-optimal dose of α-PD-1 antibody showed an over 50% reduction in MC38 tumor growth, and the effect of irisin was similar to previous results. However, the combination of irisin with α-PD-1 antibody showed a dramatic reduction in tumor growth when compared to irisin single therapy or α-PD-1 antibody when monitored until day 15 (FIG. 6B). Tumor growth was monitored until day 60 and plotted individual tumor growth curves (FIG. 6C). Three out of 8 mice from the α-PD-1 group showed complete rejection, while the control and irisin only groups failed to reject any tumors. In line with the tumor growth curve, the combination group showed an improved complete rejection rate (6 out of 8) when compared to other groups (FIG. 13A). To further verify this synergistic effect, a similar experiment was performed using an MB49 tumor model. The combination of irisin with a α-PD-1 blockade antibody showed a superior tumor-controlling effect as observed in the MC38 tumor model (FIG. 13B). Next, the human relevance of the proposed mechanisms were assessed using a publicly available dataset containing immunotherapy response rates. Expression levels of ITGAV, ITGB5, TGFB1, and FNDC5 were evaluated in two categories. Complete response (CR) and partial response (PR) groups were combined to represent immunotherapy-responsive patients, and stable disease (SD) and progressive disease (PD) groups were categorized as immunotherapy-nonresponsive patients. ITGB5 and TGFB1 expression levels were higher in the SD/PD group when compared to the CR/PR group and ITGAV showed a trend. FNDC5 showed minimal expression in this dataset (FIG. 6D). These results show that proposed mechanisms for effect of irisin can play a critical role in PD-1/PD-L1 blocking immunotherapies.

### DISCUSSION

Herein, anti-tumor activity of the exercise-induced hormone irisin and the mechanism involved in this beneficial effect was shown. Irisin treatment reduced tumor growth in multiple tumor models and this advantageous outcome was mediated by T cells. High-dimensional flow cytometry and concomitant analysis was used to depict immunological changes caused by irisin treatment. Irisin reduced accumulation of Tregs in both early- and late-time point TME. To further understand the role of irisin in Treg biology, the characteristics of Tregs in control and irisin treated tumor samples was identified. The Treg suppressive phenotype was also decreased by irisin treatment. As Tregs can hinder cytotoxic activity of CD8⁺ T cells, next the phenotype of CD8⁺ T cells was evaluated. In line with decreased Treg frequency and suppressive function, dysfunctional CD8⁺ T cell accumulation was decreased, and the effector population was enriched by irisin treatment. To elucidate the molecular mechanism responsible for irisin's effect, the involvement of integrin αvβ5, a known binding partner of irisin, and one of the major pathways regulating L-TGF-β activation was evaluated. The integrin αvβ5 complex was specifically expressed in myeloid cell compartments in TME. Myeloid cell specific knockout of integrin av led to better tumor control, and the anti-tumor effect of irisin was abolished in this knockout mice. Also, irisin treatment resulted in less conversion of L-TGF-β into active form, and the expression level and relationship between integrin αvβ5 and the TGF-β pathway were further confirmed using publicly available human datasets. Finally, the combinatorial effect of irisin with α-PD-1 antibody and confirmed its synergistic activity was tested.

Exercise and regular physical activity have been shown to reduce the incidence of various adverse health effects. These health concerns include obesity, diabetes, cancer risk, osteoporosis, neurodegenerative diseases, cardiovascular symptoms, mental health disorders, and many more. Specifically, regarding cancer, individuals who exercise demonstrated a lower rate of cancer incidence, and cancer patients with regular exercise routines had less metastasis and exhibited better treatment outcomes. Even though the importance and beneficial role of exercise is evident, the underlying mechanism has not been extensively studied. Alterations in tumors' intrinsic signaling pathways, hormone levels, whole body metabolism, and inflammation have been shown as plausible mechanisms. Additionally, it has been reported that increased blood flow as a result of exercise may reduce the hypoxic environment in the TME, but the molecular mechanism responsible for these effects has not been fully investigated. Recent studies show that CD8⁺ T cells can play critical roles in this context, implicating the importance of immune cell populations in exercise mediated tumor control.

Exercise can affect whole body metabolism through autocrine and paracrine regulations mediated by hormones. Exercise directly impacts muscle cells, and the other parts of the body are mainly regulated by myokines, such as insulin, insulin-like growth factor-1, testosterone, TSH, and irisin. Most of these hormones have been studied for their role in tumor control, but irisin's effect has not been addressed in an immuno-oncology context. Irisin was first identified in 2012 as a muscle-secreted cytokine that converts white adipose tissue into brown/beige adipose tissue. Brown/beige adipose tissue has a higher energy expenditure level than white adipose tissue, and this conversion by irisin reduces body weight and can treat metabolic disease. Later, it was proven that the beneficial effect of irisin is mediated by integrin av, expressed in bone and fat cells. Other beneficial roles, such as neurodegenerative disease prevention and muscle regeneration, have also been reported. The role of irisin in shaping tumor immunity has not been studied in animal models, but it has been reported that irisin can reduce pancreatic tumor cell growth in an in vitro cell culture system. Therefore, the possibility of irisin being directly involved in tumor cell growth by using it to treat cell lines utilized was tested. However, no growth defects or delays were observed in the cell lines utilized in this disclosure, showing that another mechanism is involved in in vivo irisin activity.

Integrin αv complexes were identified as binding partners for irisin and are the only reported receptors reported to date. Integrin av and β integrins (β3, β5, 6, and β8) form complexes that play unique roles in biological processes. One of the main and overlapping functions of these integrin complexes is converting L-TGF-β into an active form of TGF-β. TGF-β is a multi-functional cytokine that plays an immune-suppressive role within the TME largely by promoting Treg accumulation. In turn, Tregs interfere with cytotoxic T cell function, leading to poor tumor control. Another study also indicates that αvβ8 expression in tumor cells is responsible for Treg enrichment, creating immune-suppressive TME by converting L-TGF-β into its active form. TGF-β also plays a critical role in controlling the CD8⁺ T cell exhaustion process. It has been shown that TGF-β can metabolically alter CD8⁺ T cell status, leading to the development of exhausted CD8⁺ T cell subsets. Since TGF-β exerts an immunosuppressive property, multiple strategies have been deployed to better shape tumor immunity. However, single agent targeting the TGF-β signaling pathway axis did not make outstanding progress in clinical trials, requiring further understanding of this biological process. Unlike many TGF-β pathway inhibitors, where TGF-β pathway activity is systemically reduced, irisin showed higher localization to the tumor area when compared to secondary lymphoid organs. This shows that irisin can reduce TGF-β activation specifically in the TME because the binding partner of irisin (integrin complex) is highly expressed in TME compared to other organs. Spatial transcriptomics and scRNA-seq datasets were used to confirm this finding and have experimentally validated it with flow cytometry.

CD8⁺ T cells within the TME are designed to eliminate tumor cells when activated and are well equipped with multiple strategies to repress tumor growth. This often fails due to a phenomenon called T cell exhaustion (dysfunction). This dysfunctionality is characterized by expression of inhibitory molecules, reduced self-renewal capacity, and lower effector function. Persistent antigen presentation, hypoxia, nutrient deprivation, and accumulation of suppressive cytokines such as TGF-β can initiate this process. Immunotherapy blocking the PD-1/PD-L1 pathway is known to regulate this T cell exhaustion state, leading to better tumor control. More precisely, progenitor-exhausted subsets with low-to-intermediate PD-1 expression respond to PD-1/PD-L1 blocking antibodies and repopulate to further differentiate into effector T cells. Although immunotherapy has shown unprecedented success over recent decades, most patients still do not respond to this treatment modality. For this reason, extensive research and clinical trials are ongoing to find better combinatorial strategies. Irisin showed a synergistic effect with PD-1 blockade antibodies in two different tumor models. Indeed, re-analysis of publicly available datasets show that the mechanistic parts of irisin's effect correlate with better patient outcomes.

This disclosure describes the important role of irisin in shaping tumor immunity. Irisin treatment reduced the accumulation of Tregs and led to better effector function of CD8⁺ T cells. Mechanistically, irisin utilized the integrin av complex to reduce TGF-β formation. Irisin also showed a synergistic effect when combined with α-PD-1 antibody, further showing its use in therapeutics.

### Methods and Models

Animal model and experiments. C57BL/6J (Jackson Laboratory, #000664), RAG2 knockout (Jackson Laboratory, β6.Cg-Rag2^{tm1.1Cgn}/J; #008449), and TCR β8 knockout (Jackson laboratory, B6.129P2-Tcrb^{tm1Mom} Tcrd^{tm1Mom}/J; #002122) mice were used to test anti-tumor efficacy of irisin. To create a myeloid cell-specific knockout mouse model, LysMcre (Jackson Laboratory, B6.129P2-Lyz2^{tm1(cre)Ifo}/J; #004781) mice were crossed with integrin αv^{fl/fl} mice (Jackson laboratory, B6.129P2 (Cg)-Itgav^{tm2Hyn}/J; #032297) to generate cre-negative αv^{fl/fl} mice (WT) and cre-positive αv^{fl/fl} mice (LysMKO). All tumor models utilized mice aged 7-9 weeks, both male and female sex.

Tumor cell lines. MC38 colon carcinoma (Kerafast), β16F10 melanoma (ATCC), MB49 bladder cancer (Sigma-Aldrich), and Lewis lung carcinoma (LLC; ATCC) cell lines were utilized in this study. MC38, MB49, and LLC were cultured in DMEM (GIBCO) supplemented with 10% FBS (GIBCO) and 1% penicillin/streptomycin (GIBCO) at 37° C. and 5% CO₂. β16F10 cell line was cultured in RPMI1640 (GIBCO), and supplements added to media were identical to other cell lines. Cell lines used in this disclosure were tested regularly for mycoplasma contamination.

Irisin or antibody treatment. For irisin treatment, intraperitoneal injection of 1 mg/kg/day was started 7 days after tumor injection. PBS was used as injection control. For PD-1 blockade experiment, in vivo grade anti-PD-1 (clone: 29F.1A12, BioXCell) and its matching isotype control (rag IgG2a) were purchased. On day 9 post tumor implantation, 60 μg antibody was injected per mouse by intraperitoneal injection. Antibodies were injected every 3 days for 3 total doses.

In vivo tumor challenge and single cell suspension preparation. On the day of subcutaneous tumor implant, cells were detached from culture plate and washed twice with PBS and counted to inject desirable number of cells. 1 million cells per mice were injected for MC38 tumor model, and 0.25 million cells were used for other cancer models. Cells were prepared in 100 μl PBS to contain indicated number of cells and injected under the right flank of mice. Tumor area was monitored using caliper until indicated time points. To perform downstream analysis, single-cell suspensions were prepared using isolated tumor samples, spleen, and draining lymph nodes. Isolated tumors from the mice were placed on cold PBS, and mechanically chopped to facilitate enzymatic digestion. Chopped tumor was washed with PBS and incubated in RMPI 1640 media containing collagenase I (200U/ml, Worthington) for 30-60 minutes in 37° C. depending on tumor size. Digestion mixture was then neutralized with 2% BSA solution and passed through 70 μm cell strainer (Corning). Obtained single cells were re-suspended in PBS after centrifugation (1000 RPM, 5 min at 4° C.), and subjected to downstream analysis.

Flow cytometry staining. Single-cell suspensions collected from indicated source were washed with PBS, and LIVE/DEAD fixable blue (Invitrogen) staining solution (1:1000) was applied to stain dead cells. Cells were washed twice with FACS buffer and surface molecule staining antibody cocktail was applied for 45 minutes in 4° C. After incubation, cells were washed twice with FACS buffer and FOXP3/Transcription factor staining buffer set (eBioscience) was applied to fix cells overnight. After overnight fixation, cells were washed twice in permeabilization buffer, and intracellular staining antibody cocktail was added to the cells. After 2 hours of room temperature staining, cells were washed twice with FACS buffer and data was collected using Aurora (Cytek) 5-laser flow cytometry machine.

High-dimensional flow cytometry analysis. Acquired high-dimensional flow cytometry data was uploaded to web-based software OMIQ (app.omiq.ai). Live CD45⁺, CD4⁺, and CD8+ cells were gated, and dimension reduction and clustering analysis were performed. For general immune phenotyping, opt-sne dimension reduction was utilized, and the UMAP method was applied to T cell analysis. After the dimension-reduction step, the proper number of clusters were determined using FlowSOM elbow meta-clustering analysis, and FlowSOM consensus meta-clustering analysis was performed to cluster cells with unique features. Characteristics of each cluster were evaluated using individual marker expression in opt-sne or UMAP space and validated using clustered heatmap. Statistical significance was evaluated using edgeR analysis. To further verify the results, flow cytometry data was re-evaluated using FlowJo (BD) by creating 2D plots and its concomitant statistical approach.

Cell sorting and in vitro L-TGF-β activation assay. To isolate myeloid cells from tumor samples, single cell suspensions were prepared and stained with viability dye and lineage markers including CD45, CD3, and CD11b. Myeloid cells were gated and sorted into RPMI1640 media for further analysis using BD FACS melody. Sorted myeloid cells were rested at 37° C. and 5% CO₂ for 15 minutes and indicated reagents including L-TGF-β and irisin were treated to cell culture media. After 2 hours of incubation, cell culture media were collected and spun down to remove cell particles and debris. Level of activated TGF-β were measured using ELISA kit specific for active form of TGF-β.

Publicly available data set download. The bulk RNA-seq data of bladder cancer were downloaded from research-pub.gene.com/IMvigor210CoreBiologies, to perform gene expression pattern analysis. The 167 bladder tumor samples were selected based on the “Best Confirmed Overall Response” annotation, including 15 CR (complete response), PR (partial response), SD (stable disease), and PD (progressive disease). The mouse melanoma scRNA-seq data, including normalized expression matrix and annotation files, were downloaded from www.ebi.ac.uk/gxa/sc/experiments/E-EHCA-2/downloads. The human lung cancer scRNA-seq data, including seven R objects (each object represent one major cell type) containing normalized expression matrices, were downloaded from www.ebi.ac.uk/arrayexpress/experiments/E-MTAB-6149. The human breast cancer spatial transcriptomics data, named Human Breast Cancer (Block A Section 1), were obtained from 10×official website www.10xgenomics.com/resources/datasets. Two human colon cancer spatial transcriptomics data (using ST-colon1 and ST-colon2 which were primary colon cancer without treatment) were downloaded from (www.cancerdiversity.asia/scCRLM).

scRNA-seq and Visium data pre-processing and analysis. For the two scRNA-seq data, including mouse melanoma and human lung cancer, cell clustering and Uniform Manifold Approximation and Projection (UMAP) visualization were implemented using annotated cell labels to demonstrate the separation of clusters based on the Seurat framework (PMID: 31178118) (v. 4.0.2). Then, integrin av, β1, β3, β5, β6, and β8 expression levels were visualized in UMAP space. Cells with co-expression of integrin av and β5 were defined as double-positive (DP) cells to demonstrate their distribution on different cell types. For the spatial validation, human breast cancer (Visium) was normalized by the LogCPM method based on the Seurat framework using the NormalizeData function followed by default settings. Then, we visualized DP spots on the spatial map. To further validate functional differences between DP spots and non-DP spots, the TGFb pathway gene signatures were downloaded from the GSEA database (MSigDB) (PMID: 16199517). Gene sets activity was then calculated via Ucell (PMID: 34285779), the non-parametric gene-ranking method, and compared the difference of gene sets activity between DP and non-DP spots based on the two-side Wilcoxon Rank Sum Test method. The DESeq 2 (v.1.30) normalization method was applied before the survival analysis and gene expression analysis based on bulk RNA-seq. The survival analysis was performed based on the package survival (v.3.1).

Irisin purification. pHLSec2-irisin-his was a gift from Harold Erickson (Addgene plasmid #122729) and overexpressed in HEK293 cells using lipofectamine 3000 (Invitrogen) per manufacturer's protocol. The day after transfection, the media was refreshed, and cells were further incubated for 48 hours. Then media was harvested and centrifuged to remove cell debris. Irisin was purified from the media using HisPur Cobalt spin column kit (Thermo Scientific).

Quantification and statistical analysis. Statistical differences were determined using GraphPad Prism 8 (GraphPad software). Detailed information of each analysis is indicated in figure legend, and Student's t test, one-way or two-way ANOVA was performed. Following was applied to mark significant changes in figures. (*: p<0.05, **: p<0.01, ***: p<0.001.). For high dimensional flow analysis, edgeR was utilized to detect significantly affected clusters.

It will be apparent to those skilled in the art that various modifications and variations can be made in the present disclosure without departing from the scope or spirit of the invention. Other embodiments of the disclosure will be apparent to those skilled in the art from consideration of the specification and practice of the methods disclosed herein. It is intended that the specification and examples be considered as exemplary only, with a true scope and spirit of the invention being indicated by the following claims.

TABLE 1

| Marker | Fluorochrome | Company | Catalog number |
|---|---|---|---|
| F4/80 | BUV395 | BD | 565614 |
| CD8 | BUV496 | BD | 750024 |
| PD1 | BUV737 | BD | 749422 |
| PD1 | FITC | eBioscience | 11-9985-85 |
| PDL1 | BV421 | Biolegend | 124315 |
| Ly6C | Super Bright 436 | eBioscience | 62-5932-82 |
| FoxP3 | eFluor 450 | eBioscience | 48-5773-82 |
| B220 | BV570 | Biolegend | 103237 |
| CD103 | BV711 | BD | 564320 |
| CD11c | BV750 | Biolegend | 117357 |
| NK1.1 | BV785 | Biolegend | 108749 |
| NK1.1 | BV570 | Biolegend | 108733 |
| CD25 | BB515 | BD | 564424 |
| CD3 | FITC | eBioscience | 11-0031-82 |
| CD3 | BUV737 | BD | 612771 |
| CD11b | Alexa Fluor 532 | eBioscience | 58-0112-82 |
| CD4 | PerCP-Cy5.5 | eBioscience | 45-0042-82 |
| MHCII | PerCP-eFluor 710 | eBioscience | 46-5321-82 |
| Ly6G | PE-Dazzle594 | Biolegend | 127648 |
| CD38 | PE-Cy7 | Biolegend | 102718 |
| aVb5 | Alexa Fluor 647 | BD | 565836 |
| CD206 | APC-eFluor 780 | eBioscience | 47-2061-82 |
| Ki-67 | BUV395 | BD | 564071 |
| Viability dye | LIVE DEAD Blue | Invitrogen | L23105 |
| CD27 | BUV563 | BD | 741275 |
| GITR | BUV615 | BD | 751532 |
| CD44 | BUV661 | BD | 741471 |
| Lag-3 | BUV805 | BD | 748540 |
| CD62L | BV421 | Biolegend | 104436 |
| ICOS | Super Bright 436 | eBioscience | 62-9949-82 |
| CD95 | BV480 | BD | 746755 |
| CD45 | BV510 | Biolegend | 103138 |
| Klrg1 | Pacific Orange | eBioscience | 79-5893-82 |
| VISTA | Super Bright 600 | eBioscience | 63-1083-82 |
| TIGIT | BV650 | BD | 744213 |
| Tim3 | BV711 | Biolegend | 119727 |
| CD38 | BV750 | BD | 747103 |
| T-bet | BV786 | BD | 564141 |
| EOMES | PerCP-eFluor 710 | eBioscience | 46-4875-82 |
| TOX | PE | Miltenyi Biotech | 130-120-716 |
| CTLA4 | PE-Dazzle594 | Biolegend | 106318 |
| CD69 | PE-Cy5 | Biolegend | 104510 |
| Tcf1 | PE-Cy7 | CST | 90511S |
| Slamf6 | APC | eBioscience | 17-1508-82 |
| Bcl2 | Alexa Fluor 647 | Biolegend | 633510 |
| Gzm B | Alexa Fluor 700 | Biolegend | 372222 |
| CX3CR1 | APC-Cy7 | Biolegend | 149040 |
| IFN-g | BV786 | BD | 563773 |
| TNF-a | BV421 | Biolegend | 506327 |
| p-Smad2/3 | PE | BD | 562586 |

1. A method of treating or preventing a cancer in a subject, the method comprising administering a pharmaceutically effective amount of a composition comprising irisin and a pharmaceutically acceptable carrier, wherein the composition prevents TGF-β activation in the subject relative to an untreated control.

2. The method of claim 1, wherein irisin targets an integrin αvβ5 complex.

3. The method of claim 1, wherein the composition prevents release and activation of TGF-β in the subject relative to an untreated control.

4. The method of claim 1, wherein the composition reduces tumor growth in the subject relative to an untreated control.

5. The method of claim 1, wherein the composition causes accumulation of a functional T cell in the subject relative to an untreated control.

6. (canceled)

7. The method of claim 1, further comprising administering an immune checkpoint inhibitor.

8. (canceled)

9. (canceled)

10. The method of claim 1, wherein the cancer comprises colon carcinoma, melanoma, urothelial carcinoma, lung cancer, or a cancer malignancy.

11. (canceled)

12. A method of preventing tumor growth factor β (TGF-β) activation in a tumor, the method comprising:
a. administering a composition comprising irisin and a pharmaceutically acceptable carrier to a subject with said tumor, and
b. detecting a decreased amount of an activated TGF-β in the tumor relative to an amount of the activated TGF-β in a control tumor.

13. The method of claim 12, wherein irisin targets an integrin αvβ5 complex.

14. (canceled)

15. (canceled)

16. The method of claim 12, wherein the composition causes accumulation of a functional T cell in the subject relative to an untreated control.

17. (canceled)

18. The method of claim 12, further comprising administering an immune checkpoint inhibitor.

19. (canceled)

20. (canceled)

21. The method of claim 12, wherein the tumor is derived from a cancer selected from colon carcinoma, melanoma, urothelial carcinoma, lung cancer, or a cancer malignancy.

22. The method of claim 12, wherein the method treats or prevents the cancer.

23. (canceled)

24. A method of reducing tumor growth in a subject, the method comprising administering to a subject a pharmaceutically effective amount of a composition comprising irisin and a pharmaceutically acceptable carrier, wherein the composition reduces tumor growth in the subject relative to an untreated control.

25. The method of claim 24, wherein irisin targets an integrin αvβ5 complex.

26. The method of claim 24 or 25, wherein the composition prevents release and activation of TGF-β in the subject relative to an untreated control.

27. The method of claim 24, wherein the composition causes accumulation of a functional T cell in the subject relative to an untreated control.

28. (canceled)

29. The method of claim 24, wherein the method treats or prevents a cancer.

30. The method of claim 24, further comprising administering an immune checkpoint inhibitor.

31. (canceled)

32. (canceled)

33. The method of claim 24, wherein the tumor is derived from a cancer selected from colon carcinoma, melanoma, urothelial carcinoma, lung cancer, or a cancer malignancy.

34. (canceled)
